(19) United States
(12) Patent Application Publication
(10) Pub. No.: US 2006/0025589 A1
(43) Pub. Date: Feb. 2, 2006

(54) 2-Thiohydantoine derivative compounds and use thereof for the treatment of diabetes

(76) Inventors: Jean Binet, Fontaine les Dijon (FR); Benaissa Boubia, Saint Apollinaire (FR); Evelyne Chaput, Sierentz (FR); Alan Edgar, Saint Julien (FR); Khan Ou, Hauteville Les Dijon (FR); Philippe Ratel, Ahuy (FR); Soth Samreth, Daix (FR); Didier Thomas, Saint Apollinaire (FR)

Correspondence Address: MERCHANT & GOULD PC, P.O. BOX 2903, MINNEAPOLIS, MN, 55402-0903, US

(21) Appl. No.: 10/529,817

(22) PCT Filed: Oct. 3, 2003
(86) PCT No.: PCT/FR03/02904

§ 371 Date: Mar. 31, 2005

(30) Foreign Application Priority Data
Oct. 1, 2002 (FR) 0212368
Oct. 4, 2002 (FR) 0212369
Oct. 4, 2002 (FR) 0212370

(51) Int. Cl. C07D 413/02; C07D 405/02; C07D 403/02

(52) U.S. Cl. 544/139; 544/370; 548/311.1; 548/317.1

(57) ABSTRACT

The invention relates to 2-thiohydantoin compounds selected from compounds of general formula (I): 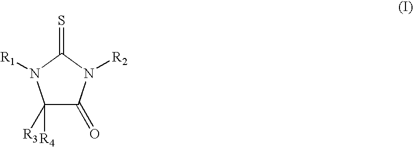
in which, in particular, one of the radicals R₁ and R₂ comprises two aromatic rings in the structure or is the dibenzofuranyl group,
R₃ is a hydrogen atom, a halogen atom, a C₁-C₄ alkyl group, a C₁-C₄ alkoxy group, a hydroxyl group, a phenyl group or a benzyl group,
R₄ is a hydrogen atom, a halogen atom or a C₁-C₄ alkyl group, and their addition salts with a non-toxic acid, especially the pharmaceutically acceptable salts. It further relates to the process for their preparation, to the pharmaceutical compositions in which they are present, and to their use as pharmacologically active substances, especially in the case of the treatment of diabetes, diseases due to hyperglycemia, hypertriglyceridemia, dyslipidemia or obesity.

The present invention relates to novel compounds derived from 2-thiohydantoin (or 2-thioxoimidazolidin-4-one), to the process for their manufacture and to their use as active principles in the preparation of drugs intended especially for the treatment of diabetes.

### PRIOR ART

The chemistry of compounds of the thiohydantoin type has been known for many years. Some derivatives of this heterocycle have been used in the field of photography, for example as described in U.S. Pat. No. 2,551,134 or JP 81 111 847, or in the field of pesticides, essentially herbicides or fungicides, for example as described in U.S. Pat. No. 3,798,233, U.S. Pat. No. 4,473,393 or the publications Indian J. Chem., 1982, vol. 21B, pp 162-164, J. Indian Chem. Soc., 1981, vol. 58 (10), pp 994-995, Chem. Abst., 67, 82381v, and Indian J. Chem., 1979, vol. 18B, pp 257-261. More recently, compounds comprising the thiohydantoin ring have been prepared for the purpose of obtaining therapeutically active products: for example, U.S. Pat. No. 3,923,994 describes the use of 3-aryl-2-thiohydantoins for their antiarthritic activity; U.S. Pat. No. 3,984,430 proposes novel thiohydantoins for the treatment of ulcers; Indian J. Chem., 1978, vol. 16B, pp 71-72, describes coumarylthiohydantoins active against tuberculosis; U.S. Pat. No. 4,312,881 claims acids and esters comprising the 2-thiohydantoin ring that have a prostaglandin-type activity; Chem. Pharm. Bull., 1982, vol. 30, no. 9, pp 3244-3254, describes the inhibition of aldose reductases by compounds of the 1-(phenylsulfonyl)-2-thiohydantoin type; Il Farmaco, Ed. Scientifico, 1983, vol. 38, no. 6, pp 383-390, proposes 3-dialkylaminopropyl-2-thiohydantoins as antiarrhythmics; WO 96/04248 describes 2-thiohydantoin derivatives of the amide or sulfonamide type that are angiotensin II antagonists; WO 97/19932 claims the use of 2-thiohydantoin derivatives for increasing HDL levels; WO 98/33776 cites a “bank” of compounds obtained by combinatorial chemistry and tested for their antimicrobial or analgesic properties; WO 93/18057 and EP 584 694 describe acids or esters comprising a 2-thiohydantoin ring that are platelet aggregation inhibitors; and EP 580 459 and WO 97/00071 propose N-phenylthiohydantoins possessing an antiandrogenic activity.

Other publications, for example J. Prakt. Chem., vol. 333(2), pp 261-266; Indian J. Chem., 1974, vol. 12, no. 6, pp 577-579; Chem. Abstr., 68, (1968) 87240d; and Organic Magn. Resonance, vol. 19 (1), pp 27-30, cite preparations of compounds comprising the 2-thiohydantoin ring without indicating the industrial utility.

The publication J. Pharm. Sc., vol. 70, no. 8, pp 952-956, cites cyclic sulfonylthiourea derivatives among which thiourea can be represented by a thiohydantoin ring, said derivatives having an antidiabetic activity at a dose of about 100 mg/kg.

### SUBJECT OF THE INVENTION

The present invention relates to novel compounds comprising the heterocycle 2-thiohydantoin (or 2-thioxoimidazolidin-4-one) in their structure, to the process for their preparation and to their use in therapeutics, especially in the preparation of a drug for the treatment of diabetes, diseases due to hyperglycemia, hypertriglyceridemia, dyslipidemia or obesity.

### DESCRIPTION

According to the invention, novel compounds are proposed that contain the 2-thioxoimidazolin-4-one (or 2-thiohydantoin) ring and are selected from:
a) compounds of the formula 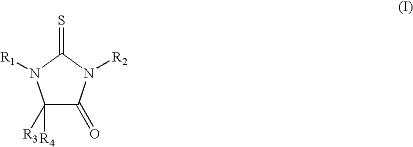
   in which
R₁ or R₂ each independently is
   a linear, branched or cyclic C₁-C₅ alkyl group,
   a C₃-C₄ alkenyl group,
   a C₂-C₃ hydroxyalkyl group or one of its precursor groups,
   a C₃-C₅ alkoxyalkyl group,
   a CH₂—COOCH₃ group,
   an N,N-dialkylaminoalkyl group,
   a group 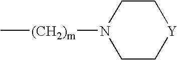
   in which m is 2 or 3 and Y is O or N—CH₃,
   a dibenzofuranyl group, or
   a group (CH₂)ₚ-Ar, in which
      p is 0 or 1, and
      Ar is a phenyl or pyridinyl aromatic ring that is unsubstituted or substituted by one or more atoms or groups of atoms selected from halogens, C₁-C₄ alkyl, hydroxyl, nitro, C₁-C₃ alkoxy, methylenedioxy, SCH₃, free or esterified carboxylic acid, trifluoromethyl, trifluoromethoxy, cyano, morpholinyl and 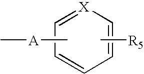
      in which
         A is O, S, CH₂, OCH₂ or CH₂O,
         X is CH or N, and
         R₅ is a hydrogen atom, a halogen atom, an N,N-dialkylamino group, a C₁-C₄ alkyl group, a C₁-C₃ alkoxy group, a hydroxyl group that is free or esterified by an amino acid, or a carboxyl or alkoxy(C₁-C₄)carbonyl group;
R₃ is a hydrogen atom, a halogen atom, a C₁-C₄ alkyl group, a C₁-C₄ alkoxy group, a hydroxyl group, a phenyl group or a benzyl group; and
R₄ is a hydrogen atom, a halogen atom or a C₁-C₄ alkyl group,
   with the proviso that at least one of the substituents R₁ and R₂ comprises in its structure 2 aromatic rings selected from phenyl and pyridinyl groups, or is the dibenzofuranyl group; and
b) addition salts of the compounds of formula (I) with a non-toxic acid if said compounds of formula (I) comprise a salifiable basic group.

In the present description, the dibenzofuranyl group is considered as comprising two aromatic rings.

One family of preferred compounds according to the invention consists of the compounds of formula (I): 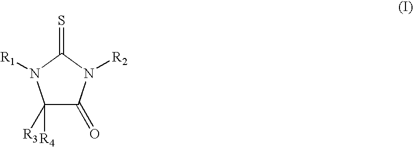
in which
R₁ and R₂ independently of one another are
   a C₁-C₅ alkyl group,
   a C₃-C₄ alkenyl group,
   a C₂-C₃ hydroxyalkyl group,
   a C₃-C₅ alkoxyalkyl group,
   a CH₂—COOCH₃ group,
   an N,N-dialkylaminoalkyl group,
   a group 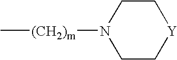
   in which m is 2 or 3 and Y is O or N—CH₃,
   a dibenzofuranyl group, or
   a group (CH₂)ₚ-Ar in which
   p is 0 or 1, and
   Ar is a phenyl or pyridinyl aromatic ring that is unsubstituted or substituted by one or more atoms or groups of atoms selected from halogens, C₁-C₄ alkyl, hydroxyl, nitro, C₁-C₃ alkoxy, methylenedioxy, ester, trifluoromethyl, trifluoromethoxy, cyano, morpholinyl and the group 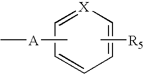
   in which
   A is O or S,
   X is CH or N, and
   R₅ is a hydrogen atom, a halogen atom, an N,N-dialkylamino group, a C₁-C₃ alkoxy group or a hydroxyl group that is free or esterified by an amino acid;
R₃ is a hydrogen atom, a halogen atom, a C₁-C₄ alkyl group, a C₁-C₄ alkoxy group, a hydroxyl group, a phenyl group or a benzyl group; and
R₄ is a hydrogen atom, a halogen atom or a C₁-C₄ alkyl group,
   with the proviso that at least one of the substituents R₁ and R₂ comprises in its structure 2 aromatic rings selected from phenyl and pyridinyl groups, or is the dibenzofuranyl group;
   and addition salts of the compounds of formula (I) with a non-toxic acid if said compounds of formula (I) comprise a salifiable basic group.

Among these compounds, very particularly preferred compounds are those of formula (I): 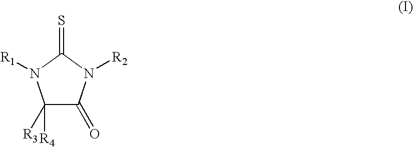
in which
R₁ is
   a C₃-C₄ alkenyl group,
   a dibenzofuranyl group, or
   a group (CH₂)ₙ-Ar in which
      n is 0 or 1, and
      Ar is a phenyl or pyridinyl aromatic ring that is unsubstituted or substituted by one or more atoms or groups of atoms selected from halogens, C₁-C₄ alkyl, nitro, C₁-C₃ alkoxy, C₃-C₄ alkoxyalkyl and the group 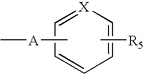
      in which
      A is O or S,
      X is C or N, and
      R₅ is a hydrogen atom, a halogen atom, an N,N-di(C₁-C₃)alkylamino group, a C₁-C₃ alkoxy group or a hydroxyl group that is free or esterified by an amino acid;
R₂ is
   a C₁-C₅ alkyl group,
   a C₃-C₄ alkenyl group,
   a C₂-C₃ hydroxyalkyl group,
   a C₃-C₅ alkoxyalkyl group,
   a CH₂—COOCH₃ group,
   a group N,N-di(C₁-C₃)alkylamino(C₁-C₃)alkyl,
   a group 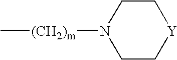
   in which m is 2 or 3 and Y is O or N—CH₃, or
   a group (CH₂)ₚ-Ar, in which
      p is 0 or 1, and
      Ar is a phenyl or pyridinyl aromatic ring that is unsubstituted or substituted by one or more atoms or groups of atoms selected from halogens, C₁-C₄ alkyl, hydroxyl, nitro, C₁-C₃ alkoxy, methylenedioxy, ester, trifluoromethyl, trifluoromethoxy, cyano, morpholinyl and the group 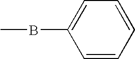
      in which
      B is O or S;
R₃ is a hydrogen atom, a halogen atom, a C₁-C₄ alkyl group, a C₁-C₄ alkoxy group, a hydroxyl group, a phenyl group or a benzyl group; and
R₄ is a hydrogen atom, a halogen atom or a C₁-C₄ alkyl group,
   with the proviso that at least one of the substituents R₁ and R₂ comprises in its structure 2 aromatic rings selected from phenyl and pyridinyl groups, or R₁ is the dibenzofuranyl group.

Another family of preferred compounds according to the invention consists of the compounds of formula (I): 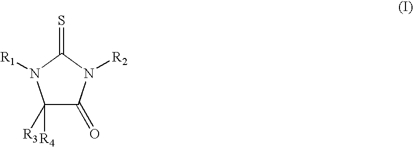
in which
R₁ and R₂ independently of one another are
   a C₁-C₅ alkyl group,
   a C₃-C₄ alkenyl group, or
   a group —(CH₂)ₙ-Ar in which
      n is 0 or 1, and
      Ar is a phenyl ring that is unsubstituted or substituted by one or more atoms or groups of atoms selected from halogens, C₁-C₄ alkyl, nitro, C₁-C₃ alkoxy, methylenedioxy, carboxyl or alkoxy(C₁-C₄)carbonyl, and 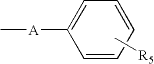
      in which
         A is CH₂O or OCH₂, and
         R₅ is a hydrogen atom, a halogen atom, a C₁-C₄ alkyl group, a C₁-C₃ alkoxy group or a carboxyl or alkoxy(C₁-C₄)carbonyl group; and
R₃ and R₄ each independently are a hydrogen atom or a C I—C₄ alkyl group,
   with the proviso that at least one of the substituents R₁ and R₂ comprises 2 aromatic rings in its structure.

Among these compounds, very particularly preferred compounds are those of formula (I): 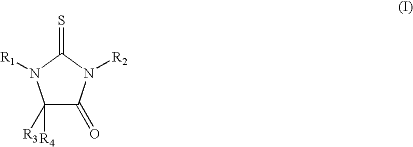
in which
R₁ is
   a C₃-C₄ alkenyl group, or
   a group —(CH₂)ₙ-Ar in which
      n is 0 or 1, and
      Ar is a phenyl ring that is unsubstituted or substituted by one or more atoms or groups of atoms selected from halogens, C₁-C₄ alkyl, nitro, C₁-C₃ alkoxy, carboxyl or alkoxy(C₁-C₄)carbonyl, and 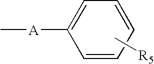
      in which
         A is CH₂O or OCH₂, and
         R₅ is a hydrogen atom, a halogen atom, a C₁-C₄ alkyl group, a C₁-C₃ alkoxy group or a carboxyl or alkoxy(C₁-C₄)carbonyl group;
R₂ is
   a C₁-C₅ alkyl group,
   a C₃-C₄ alkenyl group, or
   a group -Ar in which
      Ar is a phenyl ring that is unsubstituted or substituted by one or more atoms or groups of atoms selected from halogens, C₁-C₄ alkyl, nitro, C₁-C₃ alkoxy, methylenedioxy, carboxyl or alkoxy(C₁-C₄)carbonyl, and 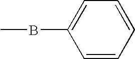
      in which
      B is CH₂O or OCH₂; and
R₃ and R₄ each independently are a hydrogen atom or a C₁-C₄ alkyl group,
   with the proviso that at least one of the substituents R₁ and R₂ comprises 2 aromatic rings in its structure.

Another family of preferred compounds according to the invention consists of the compounds of formula (I): 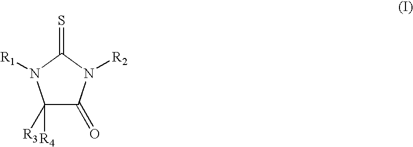
in which
R₁ and R₂ independently of one another are
   a C₁-C₅ alkyl group,
   a C₃-C₄ alkenyl group,
   a C₂-C₃ hydroxyalkyl group or one of its precursors such as a (tetrahydro-2H-pyran-2-yl)oxy(C₂-C₃)alkyl group,
   a C₃-C₅ alkoxyalkyl group, or
   a group (CH₂)ₚ-Ar in which
      p is 0 or 1, and
      Ar is a phenyl or pyridinyl aromatic ring that is unsubstituted or substituted by one or more atoms or groups of atoms selected from halogens, hydroxyl, nitro, cyano, C₁-C₃ alkoxy, carboxyl, alkoxy(C₁-C₄)-carbonyl, methylthio, methylenedioxy and 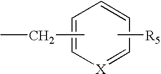
      in which
         X is CH or N, and
         R₅ is a hydrogen atom, a halogen atom, a C₁-C₃ alkoxy group or a hydroxyl group; and
R₃ and R₄ each independently are a hydrogen atom or a C I—C₄ alkyl group,
   with the proviso that at least one of the substituents R₁ and R₂ comprises in its structure 2 aromatic rings selected from phenyl and pyridinyl groups.

Among these compounds, very particularly preferred compounds are those of formula (I): 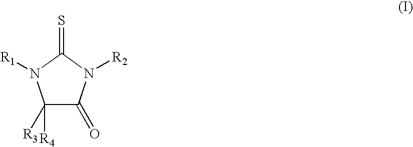
in which
R₁ is
   a C₃-C₄ alkenyl group, or
   a group (CH₂)ₙ-Ar in which
      n is 0 or 1, and
      Ar is a phenyl aromatic ring that is unsubstituted or substituted by one or more atoms or groups of atoms selected from halogens, C₁-C₃ alkoxy, nitro and the group 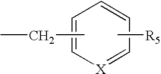
      in which
         X is CH or N, and
         R₅ is a hydrogen atom, a halogen atom, a C₁-C₃ alkoxy group or a hydroxyl group;
R₂ is
   a C₁-C₅ alkyl group,
   a C₃-C₄ alkenyl group,
   a C₂-C₃ hydroxyalkyl group or one of its precursors such as a (tetrahydro-2H-pyran-2-yl)oxy(C₂-C₃)alkyl group,
   a C₃-C₅ alkoxyalkyl group, or
   a group (CH₂)ₚ-Ar in which
      p is 0 or 1, and
      Ar is a phenyl or pyridinyl aromatic ring that is unsubstituted or substituted by one or more atoms or groups of atoms selected from halogens, hydroxyl, nitro, cyano, C₁-C₃ alkoxy, carboxyl, alkoxy(C₁-C₄)-carbonyl, methylthio, methylenedioxy and 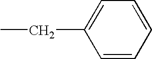
      and
R₃ and R₄ each independently are a hydrogen atom or a C₁-C₄ alkyl group,
   with the proviso that at least one of the substituents R₁ and R₂ comprises in its structure 2 aromatic rings selected from phenyl and pyridinyl groups.

Particularly preferred compounds of formula (I) according to the invention are those in which one of the radicals R₁ and R₂ is the phenoxyphenyl, phenylthiophenyl, (phenylmethoxy)phenyl or (phenylmethyl)phenyl group and the radicals R₃ and R₄ and the other radical R₁ or R₂ are as defined above.

Other preferred compounds of formula (I) are those in which R₃ is a methyl group and R₄ is a hydrogen atom or a methyl group.

In cases where the substituents R₃ and R₄ are different, the invention also includes the compounds of R configuration, the compounds of S configuration and mixtures thereof.

The invention also includes salts of the compounds of formula (I) if the latter comprise in their structure a salifiable basic group such as an amine group, a pyridine group or a morpholine group. These salts can be obtained with non-toxic and therapeutically acceptable inorganic or organic acids, especially hydrochloric, sulfuric, phosphoric, methanesulfonic, citric, maleic, fumaric, oxalic and trifluoroacetic acids.

The invention further relates to the compounds of formula (I) for their use as pharmacologically active substances.

In particular, the invention relates to the use of at least one compound of formula (I) above as an active principle in the preparation of a drug for use in therapeutics, especially for combating diseases due to hyperglycemia, diabetes, hypertriglyceridemia, dyslipidemia or obesity.

### DETAILED DESCRIPTION

In formula (I) representing the compounds according to the invention, C₁-C₄ alkyl group is understood as meaning a linear, branched or cyclic hydrocarbon chain having from 1 to 4 carbon atoms. Examples of C₁-C₄ alkyl groups include methyl, ethyl, propyl, butyl, 1-methylethyl, 1-methylpropyl, 2-methylpropyl and 1,1-dimethylethyl groups. C₁-C₅ alkyl group is understood as meaning a linear, branched or cyclic hydrocarbon chain having from 1 to 5 carbon atoms. Examples of C₁-C₅ alkyl groups include those mentioned above as well as pentyl, isopentyl and cyclopentyl groups. If a phenyl group is substituted, the substituent can be located in the ortho, meta or para position, the para position being preferred. Linear or branched C₁-C₃ alkoxy group is understood as meaning methoxy, ethoxy, propoxy and 1-methylethoxy groups.

Halogen atom is understood as meaning fluorine, chlorine, bromine and iodine atoms, fluorine and chlorine atoms being preferred.

N,N-di(C₁-C₃)alkylamino group denotes especially dimethylamino, diethylamino, dipropylamino and diisopropylamino groups.

N,N-di(C₁-C₃)alkylamino(C₁-C₃)alkyl group denotes especially dimethylaminoethyl, diethylaminoethyl and dimethylaminopropyl groups.

C₃-C₄ alkenyl group is understood as meaning a hydrocarbon chain having 3 or 4 carbon atoms that comprises an ethylenic bond between 2 carbons in its structure.

C₃-C₄ alkoxyalkyl group is understood as meaning a hydrocarbon chain having 3 or 4 carbon atoms that is interrupted by an oxygen atom, especially methoxyethyl and ethoxyethyl groups.

Precursor group of a hydroxyalkyl group is understood as meaning a group that is easily capable of generating a hydroxyalkyl group, either by means of a conventional chemical reaction (for example hydrolysis) or by means of a biological reaction (for example enzymatic hydrolysis). An example of such a precursor group is a hydroxyalkyl group protected by a tetrahydro-2H-pyran-2-yl group, which can be hydrolyzed in an acidic medium to give the corresponding hydroxylated derivative.

The compounds of formula (I) can be prepared by a first general process A comprising steps which consist in:
1) reacting an acid of the formula 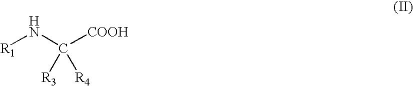
   in which R₁ is as defined above for the compounds of formula (I), R₃ is H, C₁-C₄ alkyl, phenyl or benzyl and R₄ is H or alkyl,
   with an isothiocyanate of the formula
   R₂—N═C═S (III)
   in which R₂ is a group as defined above for the compounds of formula (I), in a solvent such as ethanol, at a temperature between 20° C. and the boiling point of the solvent, in the presence of an aprotic base such as triethylamine, for 1 to 20 hours, to give the compound of formula (I): 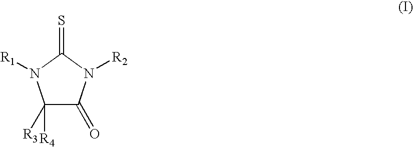
   in which R₁, R₂, R₃ and R₄ are as defined for the starting materials; and
b) if necessary, if the compound of formula (I) obtained above contains a salifiable basic group such as an amine, reacting said compound with a mineral or organic acid, in an anhydrous solvent, to give the salt of the compound of formula (I).

In one variant of this process, the acid of formula (II) can be replaced by an ester of formula (IV): 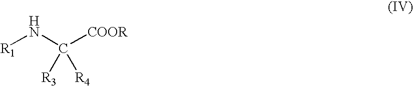
in which R₁, R₃ and R₄ are as defined in process A and R is a C₁-C₄ alkyl group, preferably a methyl, ethyl or isopropyl group, which is reacted with an isothiocyanate of formula (III):
R₂—N═C═S (III)
the reaction then being carried out in a solvent such as toluene or xylene, in the presence of a weak organic acid such as acetic acid, at a temperature between 80° C. and the boiling point of the solvent, for 0.5 to 5 hours, to give the compound of formula (I): 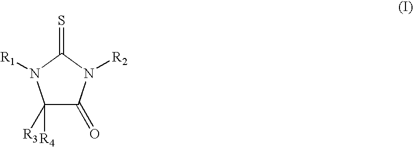
in which R₁, R₂, R₃ and R₄ are as defined for the starting compounds. This process will hereafter be called process E.

The compounds of formula (I) in which R₃ is a halogen atom, especially the fluorine atom, can be obtained from compounds of formula (I) in which R₃ is a hydrogen atom by successive reaction with a halogenating agent such as N-bromosuccinimide, water (enabling the compound of formula (I) in which R₃ is a hydroxyl group to be obtained) and then a halogenating agent such as sulphur N,N-diethylamino trifluoride, to give the compound of formula (I) in which R₃ is a fluorine atom.

The compounds of formula (I) in which R₃ is a C₁-C₄ alkoxy group can be obtained from the compounds of formula (I) in which R₃ is a hydrogen atom by reaction with a halogenating agent such as N-bromosuccinimide, followed by reaction with a C₁-C₄ aliphatic alcohol.

The compounds of formula (II) are generally known products or can be prepared by methods known to those skilled in the art, for example by reacting an aliphatic or aromatic primary amine of formula (V):
R₁—NH₂ (V)
in which R₁ is as defined above, with a halogenated acid of formula (VI): 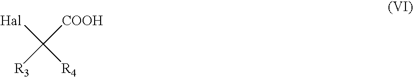
in which Hal is a halogen atom and R₃ and R₄ are as defined above, preferably in the absence of a solvent, in the presence of a weak base such as sodium bicarbonate, at a temperature of between 60 and 150° C., for 0.5 to 10 hours.

It is preferable to use an α-brominated acid.

The compounds of formula (IV) are generally known products or can be prepared by methods known to those skilled in the art, for example by reacting an aliphatic or aromatic primary amine of formula (V):
R₁—NH₂ (V)
in which R₁ is as defined above, with a halogenated ester of formula (VII): 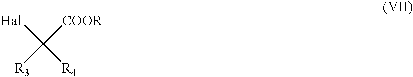
in which Hal is a halogen atom, R₃ and R₄ are as defined above and R is an alkyl group, especially methyl or ethyl, preferably in the absence of a solvent, in the presence of a weak base such as sodium bicarbonate or a tertiary amine, at a temperature of between 60 and 150° C., for 0.5 to 10 hours.

It is preferable to use an α-brominated ester.

The compounds of formula (III) are generally known products or can be prepared by methods known to those skilled in the art, for example by reacting an aliphatic or aromatic primary amine of the formula R₂—NH₂ with thiophosgene, in the presence of a tertiary amine, or with 1,1′-thiocarbonyldiimidazole.

The following Examples of the preparation of compounds of formula (I) will provide a better understanding of the invention.

In these Examples, “Preparation” denotes those which describe the synthesis of intermediates, and “Examples” denotes those which describe the synthesis of compounds of formula (I) according to the invention. The melting points are measured on a Kofler bench and the nuclear magnetic resonance spectral values are characterized by the chemical shift calculated relative to TMS, by the number of protons associated with the signal and by the shape of the signal (s for singlet, d for doublet, t for triplet, q for quadruplet, m for multiplet). The operating frequency and the solvent used are indicated for each compound.

If the compounds comprise an asymmetric carbon, the absence of a specific symbol means that the compound is in its racemic form, and the presence of the chirality symbol (R or S) means that the compound is in its chiral form.

Preparation I

### N-(4-phenoxyphenyl)alanine

203.7 g (1.1 mol) of 4-phenoxyaniline and 323 g (3.84 mol) of sodium bicarbonate are intimately mixed by grinding in a mortar. The mixture is then placed in a 2 l reactor equipped with a robust stirrer, and 306 ml (3.3 mol) of 2-bromopropionic acid are added. The mixture is heated at 90° C. for 1 hour, with stirring, and then cooled and poured into 2 l of cold water. The hydrolysis medium is then acidified slowly to pH 4 with concentrated hydrochloric acid. The precipitate formed is filtered off, washed several times with water on the filter and then dried in a vacuum oven.

This gives 178.5 g of the expected product in the form of a white solid (yield=63%).

M.p.=160° C.

Preparation II

### 1-Isothiocyanato-4-(phenylthio)benzene

A solution of 10 g (50 mmol) of 4-(phenylthio)aniline in 40 ml of dimethylformamide is prepared and a solution of 10.8 g (55 mmol) of 1,1′-thiocarbonyldiimidazole in 35 ml of dimethylformamide is added at 0° C., with stirring. The reaction medium is stirred for 5 h at 5° C. and then poured into iced water. The mixture obtained is extracted twice with 180 ml of dichloromethane and the combined organic phases are washed with water and then dried over sodium sulfate and concentrated under reduced pressure. The residue is purified by chromatography using cyclohexane as the eluent to give the expected product in the form of a colorless oil (yield=96%).

Preparation III

### N-[4-(4-fluorophenoxy)phenyl]alanine

A procedure analogous to that of Preparation I is followed, except that 4-(4-fluorophenoxy)aniline is used as the starting material, to give the expected product, which is subsequently used without further purification (yield=88%).

Preparation IV

### N-[4-(4-hydroxyphenoxy)phenyl]alanine

A procedure analogous to that of Preparation I is followed, except that 4-(4-aminophenoxy)phenol is used as the starting material, to give the expected product in the form of a fine white solid (yield=75%).

M.p.=188° C.

Preparation V

### N-[4-(phenylthio)phenyl]alanine

A procedure analogous to that of Preparation I is followed, except that 4-(phenylthio)aniline is used as the starting material, to give the expected product in the form of a light yellow oil (yield=81%).

¹H NMR (300 MHz, DMSO): 7.24 (m, 4H); 7.11 (t, 1H); 7.03 (d, 2H); 6.61 (d, 2H); 3.98 (q, 1H); 1.39 (d, 3H).

Preparation VI

### Ethyl 2-[(4-phenoxyphenyl)amino]butanoate

5 g (27 mmol) of 4-phenoxyaniline and 10.72 g (55 mmol) of ethyl 2-bromobutanoate are mixed and 3.36 g (40 mmol) of sodium bicarbonate are added. The mixture is stirred for 5 h at 140° C. and then cooled and taken up with 70 ml of water and 150 ml of ethyl ether. After decantation, the aqueous phase is re-extracted with 75 ml of ethyl ether. The combined organic phases are washed with water and then dried over sodium sulfate and concentrated under reduced pressure. The residue is purified by chromatography on silica gel using a methylcyclohexane/ethyl acetate mixture (8/2; v/v) as the eluent to give the expected product in the form of a yellow oil (yield=80%).

¹H NMR (300 MHz, DMSO): 7.29 (m, 2H); 7.01 (t, 1H); 6.83 (m, 4H); 6.59 (d, 2H); 5.91 (d, 1H); 4.11 (m, 2H); 3.86 (q, 1H); 1.78 (m, 2H); 1.17 (t, 3H); 0.97 (t, 3H).

Preparation VII

### 2-Methyl-N-(4-phenoxyphenyl)alanine

A solution of 15 g (67.7 mmol) of 4-phenoxyaniline hydrochloride in 200 ml of dimethylformamide is prepared and 13.7 g (82 mmol) of 2-bromo-2-methylpropionic acid are added, followed by 9.5 ml (67.7 mmol) of triethylamine. The reaction mixture is stirred for 24 h at 100° C. and then cooled and poured into 250 ml of iced water. The mixture is extracted with 2 times 250 ml of ethyl acetate and the combined organic phases are washed with water and then dried over sodium sulfate and concentrated under reduced pressure. The residue is purified by chromatography on silica gel to give the expected product in the form of white crystals (yield=75%).

M.p.=192° C.

Preparation VIII

### 2-Methyl-N-(2-propenyl)alanine methyl ester

15 ml of allylamine and 12 g of methyl 2-bromo-2-methylpropionate are mixed and the mixture is heated at 80° C. overnight. The excess amine is driven off under reduced pressure and the residue is purified by chromatography on silica gel using a cyclohexane/ethyl acetate mixture (7/3; v/v) as the eluent to give the expected product in the form of a pale yellow oil (yield=11%).

¹H NMR (DMSO d₆, 300 MHz): 1.20 (s, 6H); 3.03 (s, 2H); 3.62 (s, 3H); 4.98 (d, 1H); 5.11 (d, 1H); 5.79 (m, 1H).

Preparation IX

### N-(4-phenoxyphenyl)phenylalanine

A mixture of 2.17 g (7.3 mmol) of 4-phenoxyiodobenzene, 1.02 g (6.2 mmol) of phenylalanine, 0.48 g of bis(tri-o-tolylpalladium) dichloride, 125 mg of cuprous iodide, 240 mg of benzyltriethylammonium chloride and 876 mg of potassium carbonate in 12 ml of dimethylformamide, 1.2 ml of water and 2.4 ml of triethylamine is prepared. This reaction medium is stirred at 100° C. for 24 h and then cooled. 50 ml of toluene are added and the mixture is concentrated under reduced pressure. The residue is taken up with 40 ml of ethyl acetate and 40 ml of water and the mixture is acidified to pH 2. The precipitate formed is filtered off, washed with 10 ml of water and 5 ml of ethyl acetate and then dried to give 640 mg of the expected product in the form of a fine gray solid (yield=30%).

M.p.=194° C.

Preparation X

### N-[4-(4-fluorophenoxy)phenyl]alanine ethyl ester

A procedure analogous to that of Preparation VII is followed, except that 4-(4-fluorophenoxy)aniline and ethyl 2-bromopropionate are used as the starting materials in ethanol and in the presence of sodium acetate, to give the expected product in the form of a beige oily liquid, which is subsequently used without further purification (yield=80%).

Preparation XI

### N-[4-(3-chlorophenoxy)phenyl]alanine

A solution of 0.8 g (3.64 mmol) of 4-(3-chlorophenoxy)aniline in 10 ml of dimethoxyethane is prepared and 0.328 ml (3.64 mmol) of 2-bromopropionic acid and 0.5 ml of triethylamine are added. The reaction medium is stirred for 24 h at 50° C. and then cooled and poured into 50 ml of water. The mixture is brought to basic pH by adding sodium hydroxide solution, and extracted with 50 ml of ethyl acetate. The aqueous phase is then acidified to pH 4 with hydrochloric acid solution and extracted with 2 times 70 ml of ethyl ether. The combined organic phases are washed with water and then dried over magnesium sulfate and concentrated under reduced pressure to give 0.75 g of the expected product in the form of a beige solid, which is subsequently used without further purification (yield=70%).

M.p.=138-140° C.

Preparation XII

### N-[4-(2-chlorophenoxy)phenyl]alanine

A procedure analogous to that of Preparation XI is followed, except that 4-(2-chlorophenoxy)aniline is used as the starting material, to give the expected product in the form of an oil (yield=70%). This compound is subsequently used without further purification.

Preparation XIII

### N-[4-[3-(dimethylamino)phenoxy]phenyl]alanine ethyl ester

A procedure analogous to that of Preparation VII is followed, except that 4-[3-(dimethylamino)phenoxy]aniline is used as the starting material, to give the expected product in the form of a brown oil (yield=64%).

¹H NMR (300 MHz, CDCl₃): 7.18 (t, 1H); 6.89 (q, 2H); 6.60 (q, 2H); 6.41 (m, 2H); 6.23 (2d, 1H); 4.21 (q, 2H); 4.06 (q, 1H); 2.91 (s, 6H); 1.47 (d, 3H); 1.26 (t, 3H).

Preparation XIV

### N-[(4-phenoxyphenyl)methyl]alanine ethyl ester

A procedure analogous to that of Preparation VII is followed, except that 4-phenoxybenzenemethanamine and ethyl 2-bromopropionate are used as the starting material in dioxane, to give the expected product in the form of a beige oil (yield=37%).

¹H NMR (300 MHz, DMSO): 7.37 (m, 4H); 7.12 (t, 1H); 6.97 (m, 4H); 4.09 (q, 2H); 3.63 (2d, 2H); 3.24 (q, 1H); 1.20 (m, 6H).

Preparation XV

### N-(2-phenoxypyridin-5-yl)alanine

A procedure analogous to that of Preparation I is followed, except that 5-amino-2-phenoxypyridine and 2-bromopropionic acid are used as the starting materials, to give the expected product in the form of a poorly crystallized solid, which is subsequently used without further purification.

Preparation XVI

### N-[4-(4-chlorophenoxy)phenyl]alanine ethyl ester

A procedure analogous to that of Preparation X is followed, starting from 4-(4-chlorophenoxy)aniline, to give the expected product in the form of a white solid (yield=78%).

M.p.=156° C.

Preparation XVII

### N-[4-(phenylthio)phenyl]glycine

A procedure analogous to that of Preparation I is followed, starting from 4-(phenylthio)aniline and bromoacetic acid, to give the expected product in the form of an oil (yield=93%).

¹H NMR (DMSO d₆, 250 MHz): 4.13 (s, 2H); 6.61 (d, 2H); 7.09 (d, 2H); 7.30 (m, 5H).

Preparation XVIII

### 2-Methyl-N-[4-(phenylthio)phenyl]alanine

A procedure analogous to that of Preparation XVII is followed, starting from 2-bromo-2-methylpropionic acid, to give the expected product in the form of an oil (yield=99%).

¹H NMR (DMSO d₆, 300 MHz): 1.40 (s, 6H); 6.57 (d, 2H); 7.02 (d, 2H); 7.18 (m, 5H).

Preparation XIX

### N-(4-phenoxyphenyl)-2-phenylglycine

A procedure analogous to that of Preparation XI is followed to give the expected product in the form of a white solid (yield=67%).

M.p.=145° C.

Preparation XX

### 2-[(4-Phenoxyphenyl)amino]pentanoic acid

A procedure analogous to that of Preparation I is followed to give the expected product in the form of a paste (yield=70%).

¹H NMR (DMSO d₆, 300 MHz): 0.91 (t, 3H); 1.43 (m, 2H); 1.69 (m, 2H); 3.81 (t, 1H); 6.59 (d, 2H); 6.83 (m, 4H); 6.99 (t, 1H); 7.29 (t, 2H).

Preparation XXI

### Ethyl 1-[(4-phenoxyphenyl)amino]cyclopropanecarboxylate

a) A suspension of 6.15 g of 1-aminocyclopropanecarboxylic acid in 100 ml of ethanol is prepared and 6.5 ml of thionyl chloride are added gradually. The reaction mixture is refluxed gently for 8 hours and then concentrated under reduced pressure, toluene being added to drive off the ethanol. This gives 10 g of the hydrochloride of the ethyl ester of the starting acid.

b) 1.25 g of the ester hydrochloride obtained above are mixed with 6.25 g of diacetyltri(4-phenoxyphenyl)bismuth in 20 ml of dichloromethane, and 1.1 ml of triethylamine and 22 mg of copper powder are added. The reaction mixture is stirred at room temperature overnight and then chromatographed on silica gel using a dichloromethane/cyclohexane mixture (8/2; v/v) as the eluent to give 0.47 g of the expected product (yield=24%).

M.p.=80° C.

Preparation XXII

### 1-[(4-Phenoxyphenyl)amino]cyclopropanecarboxylic acid

0.35 g of the ester obtained according to Preparation XXI, 1 ml of 10% sodium hydroxide solution, 20 ml of dimethoxyethane and 20 ml of methanol are mixed and this reaction medium is stirred at room temperature overnight. This mixture is then concentrated under reduced pressure and taken up with 20 ml of water. The solution obtained is filtered and acidified with N hydrochloric acid solution. The precipitate is extracted with dichloromethane and the organic phase obtained is dried over magnesium sulfate and then concentrated to give the expected acid in the form of white crystals (yield=97%).

M.p.=163° C.

### Preparation XXIII

### 2-[(4-Phenoxyphenyl)amino]-4-methylpentanoic acid

A procedure analogous to that of Preparation I is followed, starting from 2-bromo-4-methylpentanoic acid, to give the expected product in the form of a paste (yield=10%).

¹H NMR (CDCl₃, 250 MHz): 0.9 (m, 6H); 1.6 (m, 2H); 1.8 (m, 1H); 3.8 (t, 1H); 6.6 (d, 2H); 6.9 (m, 4H); 7.0 (t, 1H); 7.3 (t, 1H).

Preparation XXIV

### N-(2,6-dimethylphenyl)-2-methylalanine

A procedure analogous to that of Preparation XI is followed to give the expected product in the form of beige crystals (yield=53%).

M.p.=148° C.

Preparation XXV

### N-[4-(phenylmethoxy)phenyl]alanine ethyl ester

A solution of 15 g (63.6 mmol) of 4-(phenylmethoxy)aniline hydrochloride in 200 ml of dimethylformamide is prepared and 13.8 g (76.4 mmol) of ethyl 2-bromopropionate are added, followed by 8.9 ml (63.6 mmol) of triethylamine. The reaction mixture is stirred for 24 h at 100° C. and then cooled and poured into 200 ml of iced water. The mixture is extracted with 2 times 200 ml of ethyl acetate and the combined organic phases are washed with water and then dried over sodium sulfate and concentrated under reduced pressure. The residue is purified by chromatography on silica gel using a cyclohexane/ethyl acetate mixture (95/5; v/v) as the eluent to give 10 g of the expected product in the form of an oil, which turns to beige crystals (yield=52%).

M.p.=70° C.

Preparation XXVI

### N-[4-(phenylmethoxy)phenyl]alanine

1 g (3.34 mmol) of the ester obtained according to Preparation XXV is dissolved in 30 ml of dimethoxyethane, and 6.7 ml (6.7 mmol) of normal sodium hydroxide solution are added. The reaction mixture is stirred for 18 h at room temperature and then partially concentrated under reduced pressure. The residue is taken up with 10 ml of water and then acidified to pH 4 with dilute hydrochloric acid. The white solid which has precipitated is filtered off, rinsed with 3 ml of water and then dried under vacuum to give 0.68 g of the expected product in the form of a fine white powder (yield=75%).

M.p.=202° C.

Preparation XXVII

### N-[4-(phenylmethoxy)phenyl]glycine ethyl ester

A procedure analogous to that of Preparation XXV is followed, except that 4-(phenylmethoxy)aniline hydrochloride and ethyl bromoacetate are used as the starting materials, to give the expected product in the form of beige crystals (yield=79%).

M.p.=70° C.

Preparation XXVIII

### Methyl 2-methyl-2-[[4-(phenylmethoxy)phenyl]amino]propionate

3 g (15 mmol) of 4-(phenylmethoxy)aniline and 5.5 g (30 mmol) of methyl 2-bromo-2-methylpropionate are mixed and 1.95 g of sodium bicarbonate are added. The reaction medium is stirred for 5 h at 140° C. and then cooled and taken up with 50 ml of water and 100 ml of ethyl ether. The aqueous phase is separated off and re-extracted with 50 ml of ethyl ether and the combined organic phases are washed with water and then dried over sodium sulfate and concentrated under reduced pressure. The residue is purified by chromatography on silica gel using a cyclohexane/ethyl acetate mixture (8/2; v/v) as the eluent to give the expected product in the form of a beige crystalline solid (yield=75%).

M.p.<50° C.

Preparation XXIX

### 2-Methyl-2-[[4-(phenylmethoxy)phenyl]amino]propionic acid

A procedure analogous to that of Preparation XXVI is followed, except that the compound obtained according to Preparation XXVIII is used as the starting material, to give the expected product in the form of a cream-colored powder (yield=82%).

M.p.=210° C.

Preparation XXX

### Ethyl 2-[[4-(phenylmethoxy)phenyl]amino]pentanoate

A procedure analogous to that of Preparation XXVIII is followed, starting from ethyl 2-bromopentanoate, to give the expected product in the form of an oil, which crystallizes from isopropyl alcohol (yield=56%).

M.p.=68° C.

Preparation XXXI

### Ethyl 2-[[4-(phenylmethoxy)phenyl]amino]butanoate

A procedure analogous to that of Preparation XXVIII is followed, starting from ethyl 2-bromobutanoate, to give the expected product in the form of an oil (yield=76%).

¹H NMR (CDCl₃, 300 MHz): 1.00 (t, 3H); 1.23 (t, 3H); 1.81 (m, 2H); 3.92 (t, 1H); 4.15 (q, 2H); 4.98 (s, 2H); 6.58 (d, 2H); 6.83 (d, 2H); 7.34 (m, 5H).

Preparation XXXII

### Ethyl 2-[(3-fluorophenyl)amino]butanoate

A procedure analogous to that of Preparation XXVIII is followed, starting from 3-fluoroaniline and ethyl 2-bromobutanoate, to give the expected product in the form of an orange oil (yield=66%).

¹H NMR (CDCl₃, 250 MHz): 0.99 (t, 3H); 1.28 (t, 3H); 1.81 (m, 2H); 3.98 (m, 1H); 4.22 (q, 2H); 4.25 (d, 1H); 6.37 (m, 3H); 7.09 (m, 1H).

Preparation XXXIII

### 1-Isothiocyanato-4-(phenylmethyl)benzene

A solution of 5 g (27 mmol) of 4-(phenylmethyl)aniline in 20 ml of dimethylformamide is prepared and a solution of 5.77 g (29 mmol) of 1,1′-thiocarbonyldiimidazole in 20 ml of dimethylformamide is added at 0° C., with stirring. The reaction medium is stirred for 5 h at 5° C. and then poured into iced water. The mixture obtained is extracted twice with 100 ml of dichloromethane and the combined organic phases are washed with water and then dried over sodium sulfate and concentrated under reduced pressure. The residue is purified by chromatography using cyclohexane as the eluent to give the expected product in the form of an oil, which crystallizes in the refrigerator (yield=88%).

M.p.<50° C.

Preparation XXXIV

### N-[4-(phenylmethyl)phenyl]alanine ethyl ester

3 g (16.4 mmol) of 4-(phenylmethyl)aniline and 4.3 ml (32.7 mmol) of ethyl 2-bromopropionate are mixed and 2.06 g (24.6 mmol) of sodium bicarbonate are added. The mixture is stirred for 5 h at 140° C. and then cooled and taken up with 50 ml of water and 100 ml of ethyl ether. After decantation, the aqueous phase is re-extracted with 50 ml of ethyl ether. The combined organic phases are washed with water and then dried over sodium sulfate and concentrated under reduced pressure. The residue is purified by chromatography on silica gel using a methylcyclohexane/ethyl acetate mixture (8/2; v/v) as the eluent to give 4.5 g of the expected product in the form of an orange-yellow oil (yield=97%).

¹H NMR (300 MHz, DMSO): 7.17 (m, 5H); 6.91 (d, 2H); 6.45 (d, 2H); 5.80 (d, 1H); 4.07 (q, 2H); 3.96 (q, 1H); 3.75 (s, 2H); 1.34 (d, 3H); 1.14 (t, 3H).

Preparation XXXV

### N-[4-(phenylmethyl)phenyl]alanine

2 g (7 mmol) of the ester obtained according to Preparation XXXIV are dissolved in 60 ml of dimethoxyethane, and 14 ml (14 mmol) of normal sodium hydroxide solution are added. The mixture is stirred for 18 hours at room temperature and then partially concentrated under reduced pressure. The residue is taken up with 25 ml of water and then acidified to pH 4 with dilute hydrochloric acid. The white solid which has precipitated is filtered off, washed with water and then dried under reduced pressure to give the expected product in the form of a beige powder (yield=64%).

M.p.=119° C.

Preparation XXXVI

### N-[4-(phenylmethyl)phenyl]glycine

A solution of 18.3 g (0.1 mol) of 4-benzylaniline in 150 ml of dimethylformamide is prepared and 20.5 g (0.12 mol) of bromoacetic acid are added, followed by 14 ml of triethylamine. The reaction mixture is stirred for 24 hours at 100° C. and then cooled and poured into 200 ml of iced water. The mixture is extracted with 2 times 200 ml of ethyl acetate and the combined organic phases are washed and then dried over sodium sulfate and concentrated under reduced pressure. The crude product is purified by chromatography on silica gel using a cyclohexane/ethyl acetate mixture (95/5; v/v) as the eluent to give the expected compound in the form of beige crystals (yield=43%).

M.p.=148° C.

Preparation XXXVII

### 2-Methyl-N-[4-(phenylmethyl)phenyl]alanine

183 g (1 mol) of 4-benzylaniline and 294 g (3.5 mol) of sodium bicarbonate are intimately mixed in a mortar. The mixture is placed in a reactor equipped with a robust stirrer, and 600 g (3 mol) of 2-bromo-2-methylpropionic acid are added. The mixture is stirred for one hour at 90° C. and then cooled and poured into 2 l of cold water. The hydrolysis medium is acidified slowly to pH 4 with concentrated hydrochloric acid. The precipitate formed is filtered off, washed with water and dried in a vacuum oven to give the expected compound in the form of pale pink crystals (yield=83%).

M.p.=130° C.

Preparation XXXVIII

### N-[4-(4-hydroxyphenylmethyl)phenyl]glycine ethyl ester

A solution of 340 mg (1.71 mmol) of 4-[(4-aminophenyl)methyl]phenol and 0.28 ml (2.55 mmol) of ethyl bromoacetate in 10 ml of 1,2-dimethoxyethane is prepared and 0.36 ml (2.55 mmol) of triethylamine is added. The reaction mixture is refluxed gently for 1.5 hours and then concentrated under reduced pressure. The evaporation residue is purified by chromatography on silica gel using a toluene/ethyl acetate mixture (9/1; v/v) as the eluent to give the expected product in the form of a colorless oil (yield=62%).

¹H NMR (CDCl₃, 300 MHz): 1.32 (t, 3H); 3.83 (s, 2H); 3.90 (s, 2H); 4.28 (q, 2H); 6.57 (d, 2H); 6.77 (d, 2H); 7.03 (t, 4H).

Preparation XXXIX

### N-[4-(pyridin-4-ylmethyl)phenyl]alanine

A procedure analogous to that of Preparation XXXVII is followed, starting from 4-(pyridin-4-ylmethyl)aniline and 2-bromopropionic acid, to give the expected product in the form of an oil (yield=17%).

¹H NMR (DMSO d₆, 300 MHz): 1.34 (d, 3H); 3.9 (s, 2H); 3.88 (q, 1H); 6.48 (d, 2H); 6.95 (d, 2H); 7.21 (d, 2H); 8.43 (d, 2H).

### EXAMPLE 1

### 5-Methyl-1-(4-phenoxyphenyl)-3-phenyl-2-thioxoimidazolidin-4-one

A mixture of 175 g (0.68 mol) of the compound obtained according to the previous step and 104 ml of triethylamine in 2 l of ethanol is prepared. The solution obtained is filtered on a glass frit and 89.5 ml (0.75 mol) of phenyl isothiocyanate are added. The reaction mixture is stirred at room temperature for 18 hours. The white precipitate formed is filtered off and then dissolved in a dichloromethane/ethanol mixture. The solution is treated with active charcoal, filtered and partially reconcentrated on an evaporator under reduced pressure. The white precipitate obtained is filtered off, washed with ethanol and dried to give 228.3 g of the expected product in the form of white crystals (yield=89%).

M.p.=141° C.

### EXAMPLES 2 AND 3

### EXAMPLE 2

### 5(S)-methyl-1-(4-phenoxyphenyl)-3-phenyl-2-thioxoimidazolidin-4-one

### EXAMPLE 3

### 5(R)-methyl-1-(4-phenoxyphenyl)-3-phenyl-2-thioxoimidazolidin-4-one

A solution of 50 mg of the racemic compound obtained according to Example 1 in 1 ml of a hexane/dichloromethane mixture is prepared. This solution is injected into a high pressure preparative chromatography device equipped with a 250×20 mm CHIRALPACK AD 10 μm column (supplied by DAICEL). The eluent is a 75/25 hexane/isopropanol mixture with a flow rate of 10 ml/min. The compound of (S) configuration has a retention time in the order of 21 to 26 min and the compound of (R) configuration has a retention time of about 32 to 37 min. The separated compounds, recovered in solution after chromatography, are obtained by evaporation of the solvent at low temperature. This gives about 9 mg of each of the two enantiomers:
Example 2 (S enantiomer): α_{D}²³=+8° (C=1.24; CH₂Cl₂).
Example 3 (R enantiomer): α_{D}²³=−6° (C=1.02; CH₂Cl₂).
   Proof of the configuration of the two enantiomers was established by non-equivocal synthesis starting from (R)-alanine and (S)-alanine.

### EXAMPLE 4

### 5-Methyl-1,3-bis(4-phenoxyphenyl)-2-thioxoimidazolidin-4-one

A procedure analogous to that of Example 1 is followed, except that the phenyl isothiocyanate is replaced by 4-phenoxyphenyl isothiocyanate, to give the expected product in the form of an off-white solid (yield=74%).

M.p.=184-186° C.

### EXAMPLE 5

### 3-(4-Methoxyphenyl)-5-methyl-1-(4-phenoxyphenyl)-2-thioxoimidazolidin-4-one

A procedure analogous to that of Example 1 is followed, except that the phenyl isothiocyanate is replaced by 4-methoxyphenyl isothiocyanate and acetonitrile is used as the solvent medium, to give the expected product in the form of white crystals (yield=84%).

M.p.=170° C.

### EXAMPLE 6

### 5-Methyl-3-(4-nitrophenyl)-1-(4-phenoxyphenyl)-2-thioxoimidazolidin-4-one

A procedure analogous to that of Example 1 is followed, except that the phenyl isothiocyanate is replaced by 4-nitrophenyl isothiocyanate, to give the expected product in the form of an orange powder (yield=70%).

M.p.=210-212° C.

### EXAMPLE 7

### 3-(4-Hydroxyphenyl)-5-methyl-1-(4-phenoxyphenyl)-2-thioxoimidazolidin-4-one

A procedure analogous to that of Example 1 is followed, except that the phenyl isothiocyanate is replaced by 4-hydroxyphenyl isothiocyanate and acetonitrile is used as the solvent, to give the expected product in the form of a fine white solid (yield=71%).

M.p.=202-204° C.

### EXAMPLE 8

### 3-Ethyl-5-methyl-1-(4-phenoxyphenyl)-2-thioxoimidazolidin-4-one

A procedure analogous to that of Example 1 is followed, except that the phenyl isothiocyanate is replaced by ethyl isothiocyanate, to give the expected product in the form of a white solid (yield=64%).

M.p.=102° C.

### EXAMPLE 9

### 5-Methyl-1-(4-phenoxyphenyl)-3-(2-propenyl)-2-thioxoimidazolidin-4-one

A procedure analogous to that of Example 1 is followed, except that the phenyl isothiocyanate is replaced by allyl isothiocyanate, to give the expected product in the form of a beige solid (yield=58%).

M.p.=77° C.

### EXAMPLE 10

### 5-Methyl-3-(4-phenoxyphenyl)-1-phenyl-2-thioxoimidazolidin-4-one

A procedure analogous to that of Example 1 is followed, except that N-phenylalanine and 4-phenoxyphenyl isothiocyanate are used as the starting materials, to give the expected product in the form of a white powder (yield=83%).

M.p.=132° C.

### EXAMPLE 11

### 5-Methyl-1-phenyl-3-[4-(phenylthio)phenyl]-2-thioxoimidazolidin-4-one

A procedure analogous to that of Example 10 is followed, except that the isothiocyanate obtained according to Preparation II is used as the starting material, to give the expected product in the form of a white powder (yield=42%).

M.p.=136-138° C.

### EXAMPLE 12

### 1-(4-Methoxyphenyl)-5-methyl-3-(4-phenoxyphenyl)-2-thioxoimidazolidin-4-one

A procedure analogous to that of Example I is followed, except that N-(4-methoxyphenyl)alanine and 4-phenoxyphenyl isothiocyanate are used as the starting materials, to give the expected product in the form of white crystals (yield=92%).

M.p.=208-210° C.

### EXAMPLE 13

### 1-[4-(4-Fluorophenoxy)phenyl]-5-methyl-3-phenyl-2-thioxoimidazolidin-4-one

A procedure analogous to that of Example I is followed, except that the acid obtained according to Preparation III is used as the starting material, to give the expected product in the form of a white powder (yield=15%).

M.p.=145° C.

### EXAMPLE 14

### 1-[4-(4-Hydroxyphenoxy)phenyl]-5-methyl-3-phenyl-2-thioxoimidazolidin-4-one

A procedure analogous to that of Example 1 is followed, except that the compound obtained according to Preparation IV is used as the starting material, to give the expected product in the form of a white powder (yield=53%).

M.p.=106-108° C.

### EXAMPLE 15

### 1-[4-(4-Hydroxyphenoxy)phenyl]-3-(4-methoxyphenyl)-5-methyl-2-thioxoimidazolidin-4-one

A procedure analogous to that of Example 14 is followed, except that the phenyl isothiocyanate is replaced by 4-methoxyphenyl isothiocyanate, to give the expected product in the form of a fine white solid (yield=15%).

M.p.=196-198° C.

### EXAMPLE 16

### 5-Methyl-3-phenyl-1-[4-(phenylthio)phenyl]-2-thioxoimidazolidin-4-one

A procedure analogous to that of Example 1 is followed, except that the compound obtained according to Preparation V is used as the starting material, to give the expected product in the form of lightweight yellow crystals (yield=47%).

M.p.=84° C.

### EXAMPLE 17

### 1-(4-Phenoxyphenyl)-3-(2-propenyl)-2-thioxoimidazolidin-4-one

A solution of 2.71 g (10 mmol) of the ethyl ester of N-[4-phenoxyphenyl]glycine in 30 ml of xylene is prepared and 1.2 g (12 mmol) of allyl isothiocyanate and 10 ml of acetic acid are added. The reaction mixture is heated for 2 h at 110° C., with stirring, and then concentrated under reduced pressure. The residue is purified by chromatography on silica gel using dichloromethane as the eluent. The pure fraction is crystallized from ethyl ether, filtered off and dried to give the expected product in the form of yellow crystals (yield=33%).

M.p.=158-160° C.

### EXAMPLE 18

### 5-Ethyl-1-(4-phenoxyphenyl)-3-phenyl-2-thioxoimidazolidin-4-one

A procedure analogous to that of Example 17 is followed, except that the compound obtained according to Preparation VI is used as the starting material, to give the expected product in the form of a white powder (yield=43%).

M.p.=158-159° C.

### EXAMPLE 19

### 3-(4-Fluorophenyl)-5-methyl-1-(4-phenoxyphenyl)-2-thioxoimidazolidin-4-one

A mixture of 1.29 g (5 mmol) of the acid obtained according to Preparation 1 and 40 ml of acetonitrile is prepared. 1.14 ml (8.4 mmol) of triethylamine are added (giving a solution), followed by 1.15 g (7.5 mmol) of 4-fluorophenyl isothiocyanate. The reaction mixture is stirred for 15 h at room temperature and the solvent is then removed under reduced pressure. The residue is purified by chromatography on silica gel using a cyclohexane/ethyl acetate mixture (9/1; v/v) as the eluent to give the expected product in the form of a powder (yield=16%).

M.p.=150° C.

### EXAMPLE 20

### 3-(3-Fluorophenyl)-5-methyl-1-(4-phenoxyphenyl)-2-thioxoimidazolidin-4-one

A procedure analogous to that of Example 19 is followed, except that 3-fluorophenyl isothiocyanate is used as the starting material in dichloromethane, to give the expected product in the form of white crystals (yield=65%).

M.p.=116° C.

### EXAMPLE 21

### 3-(3,4-Dimethoxyphenyl)-5-methyl-1-(4-phenoxyphenyl)-2-thioxoimidazolidin-4-one

A procedure analogous to that of Example 19 is followed, except that 3,4-dimethoxyphenyl isothiocyanate is used as the starting material in dichloromethane, to give the expected product in the form of a white solid (yield=74%).

M.p.=156° C.

### EXAMPLE 22

### 3-(3,4-Methylenedioxyphenyl)-5-methyl-1-(4-phenoxyphenyl)-2-thioxoimidazolidin-4-one

A procedure analogous to that of Example 19 is followed, starting from 3,4-methylenedioxyphenyl isothiocyanate in dichloromethane, to give the expected product in the form of a white powder (yield=73%).

M.p.=185° C.

### EXAMPLE 23

### 3-Cyclopentyl-5-methyl-1-(4-phenoxyphenyl)-2-thioxoimidazolidin-4-one

A procedure analogous to that of Example 19 is followed, except that cyclopentyl isothiocyanate is used as the starting material, to give the expected product in the form of white crystals (yield=35%).

M.p.=99° C.

### EXAMPLE 24

### 3-(2-Methoxyethyl)-5-methyl-1-(4-phenoxyphenyl)-2-thioxoimidazolidin-4-one

A procedure analogous to that of Example 19 is followed, except that 2-methoxyethyl isothiocyanate is used as the starting material in ethanol, to give the expected compound in the form of an orange gummy product (yield=76%).

¹H NMR (300 MHz, CHCl₃): 7.4 (m, 2H); 7.33 (m, 2H); 7.19 (m, 1H); 7.08 (m, 4H); 4.42 (q, 1H); 4.13 (t, 2H); 3.72 (t, 2H); 3.39 (s, 3H); 1.42 (d, 3H).

### EXAMPLE 25

### 3-(2-Hydroxyethyl)-5-methyl-1-(4-phenoxyphenyl)-2-thioxoimidazolidin-4-one

A solution of 1.5 g (4.21 mmol) of the compound obtained according to Example 24 in 75 ml of dichloromethane is prepared. The mixture is cooled to −70° C. and 16.8 ml (16.8 mmol) of a normal solution of boron tribromide in dichloromethane are added. The reaction medium is stirred at −70° C. for 15 min and then at 0° C. for 2 h, after which it is poured into 500 ml of water. The mixture obtained is extracted with 500 ml of ethyl acetate. The organic phase is washed with water, dried over magnesium sulfate and concentrated under reduced pressure. The residue is purified by chromatography on silica gel using a dichloromethane/diethyl ether mixture (80/20; v/v) as the eluent to give the expected product in the form of white crystals (yield=29%).

M.p.=120° C.

### EXAMPLE 26 a

### 5-Methyl-3-[2-(morpholin-4-yl)ethyl]-1-(4-phenoxyphenyl)-2-thioxoimidazolidin-4-one

A procedure analogous to that of Example 19 is followed, except that 2-(morpholin-4-yl)ethyl isothiocyanate is used as the starting material, to give the expected product in the form of a white foam (yield=58%).

¹H NMR (300 MHz, DMSO): 7.47 (m, 4H); 7.14 (m, 5H); 4.90 (q, 1H); 3.90 (t, 2H); 3.5 (m, 4H); 2.54 (m, 6H); 1.25 (d, 3H).

### EXAMPLE 26 b

### 5-Methyl-3-[2-(morpholin-4-yl)ethyl]-1-(4-phenoxyphenyl)-2-thioxoimidazolidin-4-one hydrochloride

A solution of 0.27 g (0.656 mmol) of the compound obtained according to Example 26 a in 20 ml of diethyl ether and 2 ml of ethanol is prepared and 0.7 ml of a normal solution of hydrogen chloride in ethyl ether is added. A white precipitate forms. 25 ml of ethyl ether are added and the precipitate is then isolated by filtration. The solid is washed on the filter with 2 times 5 ml of ethyl ether and then dried to give 0.27 g of the expected product in the form of fine white crystals (yield=94%).

M.p.=246° C.

### EXAMPLE 27 a

### 5-Methyl-3-[3-(morpholin-4-yl)propyl]-1-(4-phenoxyphenyl)-2-thioxoimidazolidin-4-one

A procedure analogous to that of Example 19 is followed, except that 3-(morpholin-4-yl)propyl isothiocyanate is used as the starting material, to give the expected product in the form of a pale yellow oil (yield=61%).

¹H NMR (300 MHz, DMSO): 7.45 (m, 4H); 7.08 (m, 5H); 4.86 (q, 1H); 3.82 (t, 2H); 3.68 (m, 4H); 2.33 (m, 6H); 1.82 (m, 2H); 1.26 (d, 3H).

### EXAMPLE 27 b

### 5-Methyl-3-[3-(morpholin-4-yl)propyl]-1-(4-phenoxyphenyl)-2-thioxoimidazolidin-4-one hydrochloride

A procedure analogous to that of Example 26 b is followed, except that the compound obtained according to Example 27 a is used as the starting material, to give the expected product in the form of white crystals (yield=84%).

M.p.=140° C.

### EXAMPLE 28 a

### 5-Methyl-1-(4-phenoxyphenyl)-3-(pyridinyl)-2-thioxoimidazolidin-4-one

A procedure analogous to that of Example 19 is followed, except that pyridin-3-yl isothiocyanate is used as the starting material, to give the expected product in the form of a white foam (yield=68%).

¹H NMR (300 MHz, DMSO): 8.63 (m, 2H); 7.88 (m, 1H); 7.50 (2m, 5H); 7.17 (2m, 5H); 5.07 (q, 1H); 1.39 (d, 3H).

### EXAMPLE 28 b

### 5-Methyl-1-(4-phenoxyphenyl)-3-(pyridinyl)-2-thioxoimidazolidin-4-one hydrochloride

A procedure analogous to that of Example 26 b is followed, except that the compound obtained according to Example 28 a is used as the starting material, to give the expected product in the form of white crystals (yield=96%).

M.p.=140° C.

### EXAMPLE 29

### 5-Methyl-1-(4-phenoxyphenyl)-3-(phenylmethyl)-2-thioxoimidazolidin-4-one

A procedure analogous to that of Example 19 is followed, except that benzyl isothiocyanate is used as the starting material, to give the expected product in the form of an oil, which then crystallizes (yield=57%).

M.p.=62° C.

### EXAMPLE 30

### 5,5-Dimethyl-3-(4-methoxyphenyl)-1-(4-phenoxyphenyl)-2-thioxoimidazolidin-4-one

A procedure analogous to that of Example 19 is followed, except that the acid obtained according to Preparation VII and 4-methoxyphenyl isothiocyanate are used as the starting materials, to give the expected product in the form of white crystals (yield=32%).

M.p.=144° C.

### EXAMPLE 31

### 1-(4-Phenoxyphenyl)-3-phenyl-2-thioxoimidazolidin-4-one

A procedure analogous to that of Example 17 is followed, except that the ethyl ester of N-(4-phenoxyphenyl)glycine and phenyl isothiocyanate are used as the starting materials, to give the expected product in the form of a white powder (yield=84%).

M.p.=213° C.

### EXAMPLE 32

### 5-Methoxy-1-(4-phenoxyphenyl)-3-phenyl-2-thioxoimidazolidin-4-one

A solution of 0.4 g (1.1 mmol) of the compound obtained according to Example 31 in 60 ml of carbon tetrachloride is prepared and 0.22 g (1.22 mmol) of N-bromosuccinimide is added. The reaction medium is then stirred for 1 h at the reflux temperature of the solvent. After cooling to room temperature, 50 ml of methanol are added and the mixture is stirred for 15 min and then concentrated under reduced pressure. The residue is purified by chromatography on silica gel using dichloromethane as the eluent. The product obtained is crystallized from ethyl ether, filtered off and dried to give the expected product in the form of light orange crystals (yield=87%).

M.p.=164° C.

### EXAMPLE 33

### 5-Fluoro-1-(4-phenoxyphenyl)-3-phenyl-2-thioxoimidazolidin-4-one

A solution of 0.5 g (1.33 mmol) of the compound obtained according to Example 47 in 10 ml of dichloromethane is prepared and 0.53 ml of diethylaminosulfur trifluoride is added. The reaction mixture is stirred for 10 min and then concentrated under reduced pressure. The residue obtained is purified by chromatography on silica gel using a dichloromethane/cyclohexane mixture (6/4; v/v) as the eluent to give the expected product in the form of beige crystals (yield=63%).

M.p.=126° C.

### EXAMPLE 34

### 3,5-Diphenyl-1-(4-phenoxyphenyl)-2-thioxoimidazolidin-4-one

A procedure analogous to that of Example 19 is followed, except that α-[(4-phenoxyphenyl)amino]benzeneacetic acid and phenyl isothiocyanate are used as the starting materials, to give the expected product in the form of a white powder (yield=20%).

M.p.=100° C.

### EXAMPLE 35

### 1-(4-Phenoxyphenyl)-3-phenyl-5-phenylmethyl-2-thioxoimidazolidin-4-one

A procedure analogous to that of Example 19 is followed, except that the acid obtained according to Preparation 1× and phenyl isothiocyanate are used as the starting material, to give the expected product in the form of a fine white solid (yield=30%).

M.p.=130° C.

### EXAMPLE 36

### 1-[4-(4-Fluorophenoxy)phenyl]-3-(4-hydroxyphenyl)-5-methyl-2-thioxoimidazolidin-4-one

A procedure analogous to that of Example 17 is followed, except that the compound obtained according to Preparation X and 4-hydroxyphenyl isothiocyanate are used as the starting materials in toluene, to give the expected product in the form of white crystals (yield=30%).

M.p.=148° C.

### EXAMPLE 37

### 1-[4-(4-Fluorophenoxy)phenyl]-3-(4-methoxyphenyl)-5-methyl-2-thioxoimidazolidin-4-one

A procedure analogous to that of Example 36 is followed, except that 4-methoxyphenyl isothiocyanate is used as the starting material, to give the expected product in the form of white crystals (yield=40%).

M.p.=194° C.

### EXAMPLE 38

### 1-[4-(3-Chlorophenoxy)phenyl]-5-methyl-3-phenyl-2-thioxoimidazolidin-4-one

A procedure analogous to that of Example 1 is followed, except that the acid obtained according to Preparation XI is used as the starting material, to give the expected product in the form of a flaky white solid (yield=70%).

M.p.=156° C.

### EXAMPLE 39

### 1-[4-(2-Chlorophenoxy)phenyl]-5-methyl-3-phenyl-2-thioxoimidazolidin-4-one

A procedure analogous to that of Example 19 is followed, except that the compound obtained according to Preparation XII and phenyl isothiocyanate are used as the starting material, to give the expected product in the form of a white powder (yield=25%).

M.p.=108° C.

### EXAMPLE 40 a

### 1-[4-[3-(Dimethylamino)phenoxy]phenyl]-5-methyl-3′-phenyl-2-thioxoimidazolidin-4-one

A procedure analogous to that of Example 17 is followed, using the ester obtained according to Preparation XIII and phenyl isothiocyanate as the starting materials in toluene, to give the expected product in the form of a beige powder (yield=33%).

M.p.=135° C.

### EXAMPLE 40 b

### 1-[4-[3-(Dimethylamino)phenoxy]phenyl]-5-methyl-3-phenyl-2-thioxoimidazolidin-4-one hydrochloride

0.32 g (0.76 mmol) of the compound obtained according to Example 40 a is dissolved in 5 ml of a normal solution of hydrogen chloride in ethanol at 0° C. The solution obtained is then poured slowly into 30 ml of ethyl ether cooled to 0° C. The precipitate formed is filtered off and then dried under vacuum to give the expected product in the form of a white powder (yield=91%).

M.p.=142° C.

### EXAMPLE 41

### 1-[4-[4-[2-(Diethylamino)-1-oxoethoxy]phenoxy]phenyl]-3-(4-methoxyphenyl)-5-methyl-2-thioxoimidazolidin-4-one hydrochloride

A mixture of 1 g (2.38 mmol) of the compound obtained according to Example 15, 0.24 g of triethylamine and 0.23 g of ethyl chloroformate in 100 ml of dichloromethane is prepared. The mixture is stirred for 30 min at room temperature and 0.28 g of N,N-diethylglycine is then added. After stirring for 24 h at room temperature, the reaction mixture is poured into 50 ml of water. The organic phase is separated off and the aqueous phase is extracted with 40 ml of dichloromethane. The combined organic phases are washed with water and then dried over magnesium sulfate and concentrated under reduced pressure. The semisolid residue is taken up with 25 ml of ethyl acetate, and 2.5 ml of a normal solution of hydrogen chloride in ethyl ether are added. The precipitate obtained is filtered off, rinsed with 4 ml of ethyl ether and dried under vacuum to give the expected product in the form of pale yellow crystals (yield=96%).

M.p.=120° C.

### EXAMPLE 42

### 5-Methyl-1-(4-phenoxyphenyl)methyl-3-phenyl-2-thioxoimidazolidin-4-one

A procedure analogous to that of Example 40 a is followed, except that the ester obtained according to Preparation XIV is used as the starting material, to give the expected product in the form of white crystals (yield=86%).

M.p.=122° C.

### EXAMPLE 43

### 5-Methyl-1-(2-phenoxypyridin-5-yl)-3-phenyl-2-thioxoimidazolidin-4-one

A procedure analogous to that of Example 19 is followed, except that the acid obtained according to Preparation XV and phenyl isothiocyanate are used as the starting materials, to give the expected product in the form of white crystals (yield=25%).

M.p.=156° C.

### EXAMPLE 44

### 5-Methyl-3-(4-phenoxyphenyl)-1-phenylmethyl-2-thioxoimidazolidin-4-one

A procedure analogous to that of Example 19 is followed, except that N-(phenylmethyl)alanine and 4-phenoxyphenyl isothiocyanate are used as the starting material, to give the expected product in the form of an off-white powder (yield=50%).

M.p.=138° C.

### EXAMPLE 45

### 5-Methyl-3-(3-phenoxyphenyl)-1-(2-propenyl)-2-thioxoimidazolidin-4-one

A procedure analogous to that of Example 17 is followed, except that the ethyl ester of N-(2-propenyl)alanine and 3-phenoxyphenyl isothiocyanate are used as the starting materials, to give the expected product in the form of white crystals (yield=77%).

M.p.=88° C.

### EXAMPLE 46

### 3-(4-Nitrophenyl)-1-(4-phenoxyphenyl)-2-thioxoimidazolidin-4-one

A procedure analogous to that of Example 19 is followed, except that N-(4-phenoxyphenyl)glycine and 4-nitrophenyl isothiocyanate are used as the starting material, to give the expected product in the form of a beige powder (yield=40%).

M.p.=204° C.

### EXAMPLE 47

### 5-Hydroxy-1-(4-phenoxyphenyl)-3-phenyl-2-thioxoimidazolidin-4-one

A procedure analogous to that of Example 32 is followed, except that the brominated derivative is treated with water instead of methanol, to give the expected product in the form of pale orange crystals (yield=61%).

M.p.=160° C.

The chemical structures of compounds 1 to 47 described above are collated in Table I below.

Table II collates other Examples (48 to 137) of compounds of formula (I) in which A is O, obtained by preparative methods analogous to those used to obtain Examples 1 to 47; the letters A and E, indicating the preparative method, correspond to the processes of Example 1 (from an acid) and Example 18 (from an ester), respectively.

TABLE I

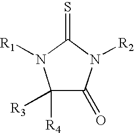

| Ex. | R₁ | R₂ | R₃ | R₄ |
|---|---|---|---|---|
| 1 | 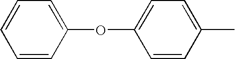 | 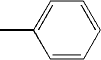 | CH₃ | H |
| 2 | 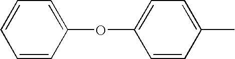 | 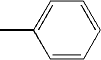 | (S) CH₃ | H |
| 3 | 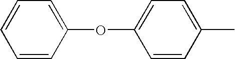 | 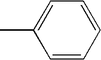 | (R) CH₃ | H |
| 4 | 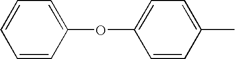 | 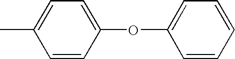 | CH₃ | H |
| 5 | 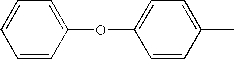 | 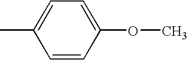 | CH₃ | H |
| 6 | 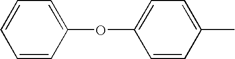 | 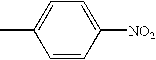 | CH₃ | H |
| 7 | 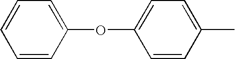 | 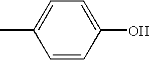 | CH₃ | H |
| 8 | 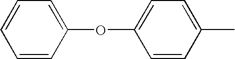 | —C₂H₅ | CH₃ | H |
| 9 | 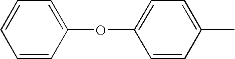 | —C₂—CH═CH₂ | CH₃ | H |
| 10 | 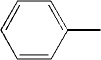 | 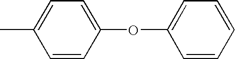 | CH₃ | H |
| 11 | 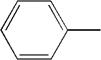 | 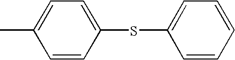 | CH₃ | H |
| 12 | 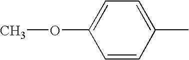 | 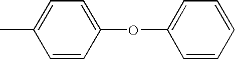 | CH₃ | H |
| 13 | 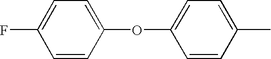 | 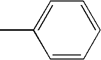 | CH₃ | H |
| 14 | 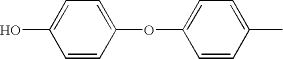 | 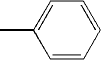 | CH₃ | H |
| 15 | 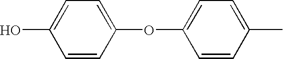 | 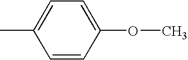 | CH₃ | H |
| 16 | 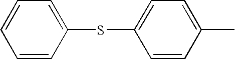 | 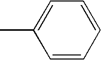 | CH₃ | H |
| 17 | 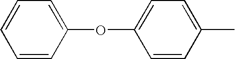 | —C₂—CH═CH₂ | H | H |
| 18 | 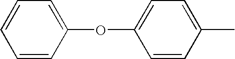 | 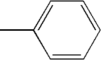 | C₂H₅ | H |
| 19 | 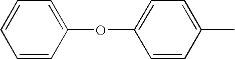 | 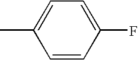 | CH₃ | H |
| 20 | 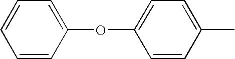 | 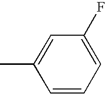 | CH₃ | H |
| 21 | 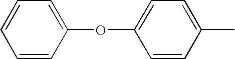 | 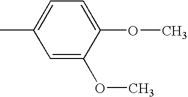 | CH₃ | H |
| 22 | 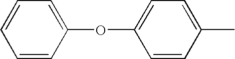 | 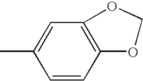 | CH₃ | H |
| 23 | 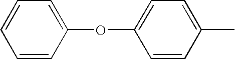 | 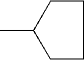 | CH₃ | H |
| 24 | 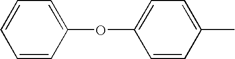 | —CH₂—CH₂—O—CH₃ | CH₃ | H |
| 25 | 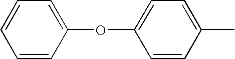 | —CH₂—CH₂—O—H | CH₃ | H |
| 26b | 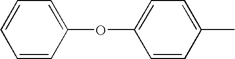 | 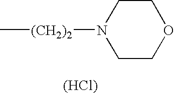 | CH₃ | H |
| 27b | 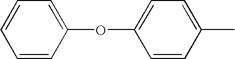 | 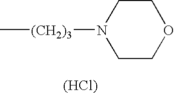 | CH₃ | H |
| 28b | 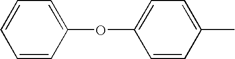 | 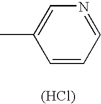 | CH₃ | H |
| 29 | 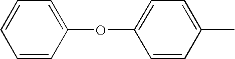 | 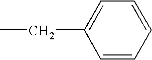 | CH₃ | H |
| 30 | 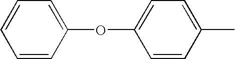 | 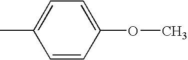 | CH₃ | CH₃ |
| 31 | 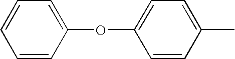 | 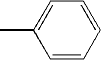 | H | H |
| 32 | 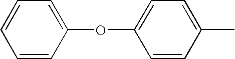 | 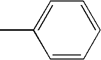 | OCH₃ | H |
| 33 | 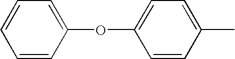 | 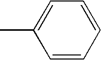 | F | H |
| 34 | 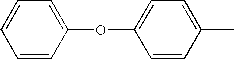 | 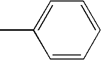 | 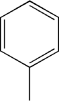 | H |
| 35 | 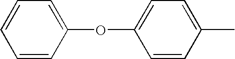 | 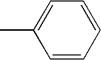 | 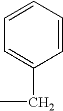 | H |
| 36 | 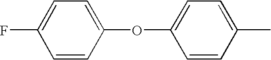 | 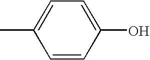 | CH₃ | H |
| 37 | 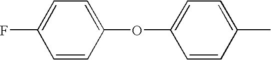 | 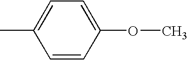 | CH₃ | H |
| 38 | 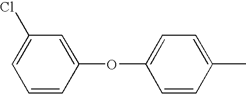 | 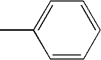 | CH₃ | H |
| 39 | 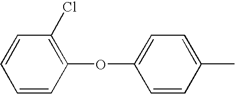 | 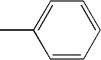 | CH₃ | H |
| 40a | 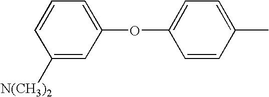 | 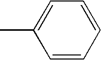 | CH₃ | H |
| 40b | 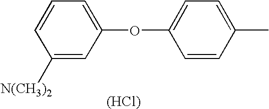 | 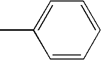 | CH₃ | H |
| 41 | 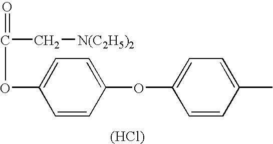 | 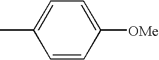 | CH₃ | H |
| 42 | 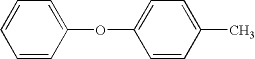 | 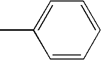 | CH₃ | H |
| 43 | 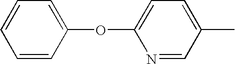 | 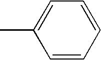 | CH₃ | H |
| 44 | 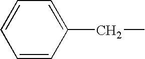 | 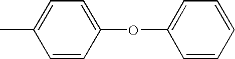 | CH₃ | H |
| 45 | CH₂═CH—CH₂— | 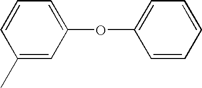 | CH₃ | H |
| 46 | 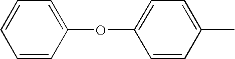 | 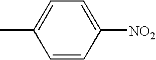 | H | H |
| 47 | 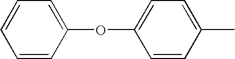 | 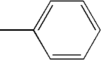 | HO | H |

TABLE II

| | | | | | M.p. | Appear- | | |
|---|---|---|---|---|---|---|---|---|
| Ex. | R₁ | R₂ | R₃ | R₄ | ° C. | ance | Yield | Meth. |
| 48 | 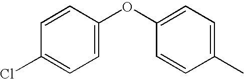 | 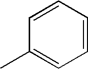 | H | —CH₃ | 168 | white powder | 62 | E |
| 49 | 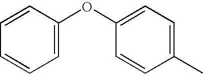 | 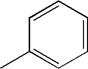 | —CH₃ | —CH₃ | 195 | white powder | 30 | E |
| 50 | 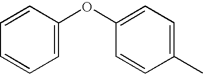 | 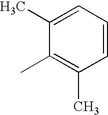 | H | —CH₃ | 204 | off-white powder | 40 | A |
| 51 | 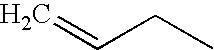 | 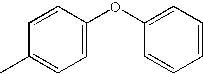 | H | —CH₃ | 130 | white powder | 51 | E |
| 52 | 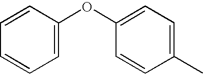 | 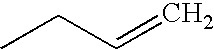 | —CH₃ | —CH₃ | 113 | off-white powder | 12 | A |
| 53 | 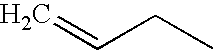 | 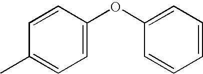 | H | H | 148 | off-white powder | 46 | E |
| 54 | 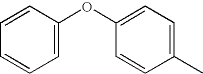 | 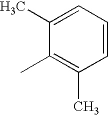 | —CH₃ | —CH₃ | 238 | white powder | 27 | A |
| 55 | 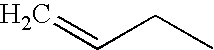 | 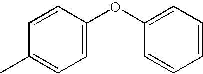 | —CH₃ | —CH₃ | 78 | white crystals | 83 | A |
| 56 | 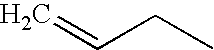 | 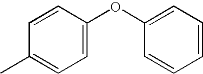 | H | —CH₃ | 157 | white powder | 75 | A |
| 57 | 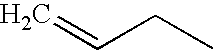 | 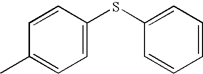 | H | —CH₃ | 100 | light white powder | 77 | E |
| 58 | 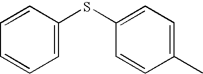 | 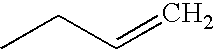 | H | —CH₃ | 108 | pale yellow solid | 28 | A |
| 59 | 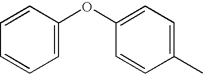 | 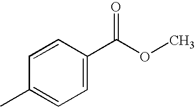 | H | —CH₃ | 144 | white powder | 86 | A |
| 60 | 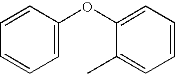 | 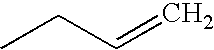 | H | —CH₃ | NMR 60 | colorless oil | 41 | A |
| 61 | 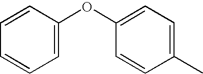 | 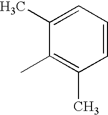 | H | —CH₃ | 174 | light white powder | 50 | A |
| 62 | 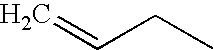 | 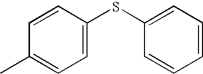 | H | H | 128 | white powder | 67 | E |
| 63 | 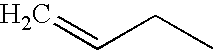 | 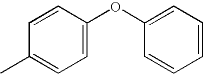 | H | H | 181 | white powder | 19 | A |
| 64 | 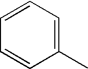 | 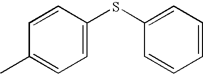 | H | H | 155 | yellow foam | 71 | A |
| 65 | 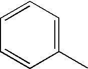 | 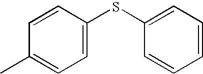 | —CH₃ | —CH₃ | 193 | white powder | 50 | A |
| 66 | 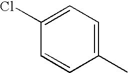 | 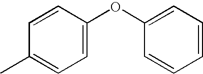 | H | —CH₃ | 141 | off-white crystals | 59 | A |
| 67 | 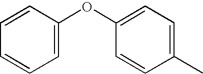 | 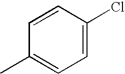 | H | H | 192 | beige powder | 57 | A |
| 68 | 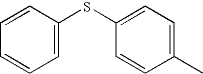 | 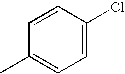 | H | —CH₃ | 120 | white powder | 49 | A |
| 69 | 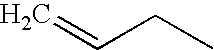 | 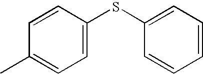 | —CH₃ | —CH₃ | 65 | white solid | 62 | E |
| 70 | 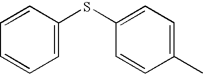 | 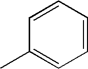 | —CH₃ | —CH₃ | 174 | white flakes | 30 | A |
| 71 | 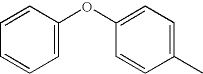 | 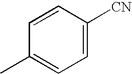 | —CH₃ | —CH₃ | 168 | white powder | 71 | A |
| 72 | 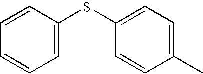 | 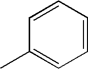 | H | H | 165 | red brown powder | 25 | E |
| 73 | 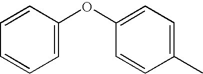 | 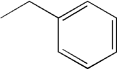 | —CH₃ | —CH₃ | 154 | white powder | 82 | A |
| 74 | 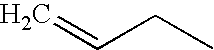 | 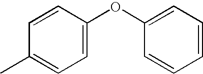 | —CH₃ | —CH₃ | 100 | white powder | 85 | E |
| 75 | 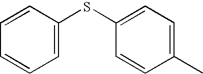 | 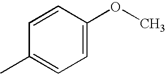 | H | —CH₃ | 150 | white powder | 47 | A |
| 76 | 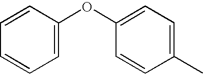 | 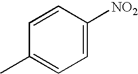 | —CH₃ | —CH₃ | 212 | white powder | 86 | A |
| 77 | 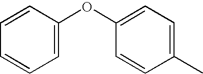 | 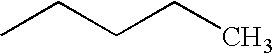 | —CH₃ | —CH₃ | 77 | pale yellow powder | 69 | A |
| 78 | 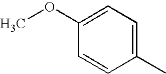 | 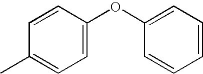 | —CH₃ | —CH₃ | 242 | white powder | 51 | A |
| 79 | 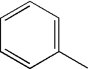 | 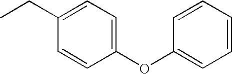 | H | H | NMR 79 | yellow foam | 96 | A |
| 80 | 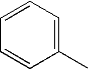 | 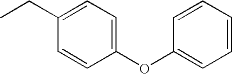 | H | —CH₃ | NMR 80 | white foam | 93 | A |
| 81 | 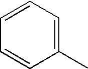 | 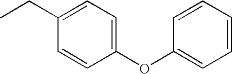 | —CH₃ | —CH₃ | NMR 81 | pale yellow foam | 40 | A |
| 82 | 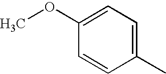 | 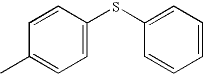 | —CH₃ | —CH₃ | 202 | white powder | 36 | A |
| 83 | 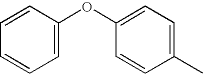 | 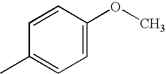 | H | H | 184 | flaky orange crystals | 60 | A |
| 84 | 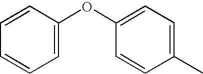 | 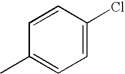 | H | —CH₃ | 185 | off-white solid | 68 | A |
| 85 | 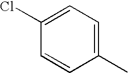 | 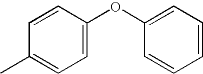 | H | H | 164 | cottony white solid | 64 | A |
| 86 | 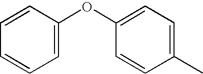 | 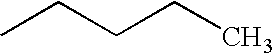 | H | H | 96 | white cotton | 50 | A |
| 87 | 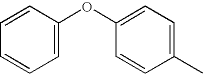 | 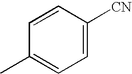 | H | H | 194.5 | beige powder | 33 | A |
| 88 | 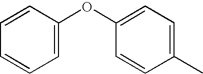 | 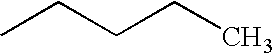 | H | —CH₃ | 206 | white solid | 50 | A |
| 89 | 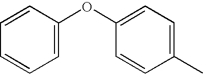 | 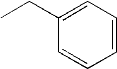 | H | H | 148 | beige cotton | 50 | A |
| 90 | 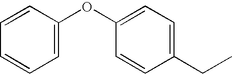 | 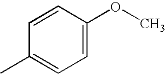 | H | —CH₃ | 134 | white powder | 78 | E |
| 91 | 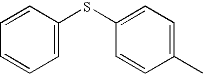 | 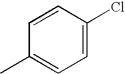 | —CH₃ | —CH₃ | 170 | white powder | 27 | A |
| 92 | 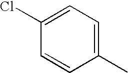 | 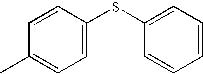 | H | —CH₃ | 132 | white powder | 50 | A |
| 93 | 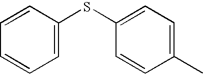 | 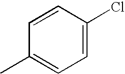 | H | H | 165 | beige powder | 38 | A |
| 94 | 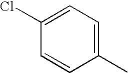 | 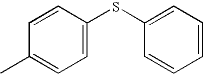 | —CH₃ | —CH₃ | 154 | white powder | 45 | A |
| 95 | 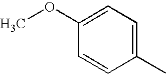 | 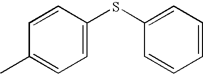 | H | —CH₃ | 194 | white powder | 53 | A |
| 96 | 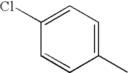 | 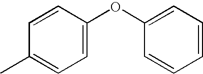 | —CH₃ | —CH₃ | 184 | white powder | 45 | A |
| 97 | 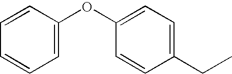 | 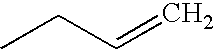 | H | —CH₃ | NMR 97 | oil | 92 | E |
| 98 | 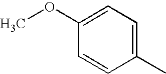 | 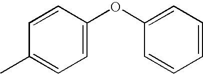 | H | H | 167 | pale yellow powder | 25 | A |
| 99 | 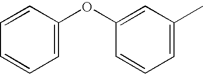 | 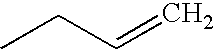 | H | —CH₃ | NMR 99 | colorless oil | 53 | A |
| 100 | 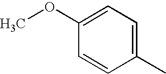 | 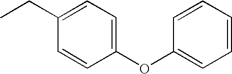 | H | —CH₃ | 99 | beige powder | 40 | A |
| 101 | 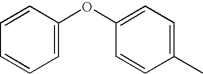 | 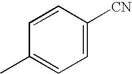 | H | —CH₃ | 184 | white powder | 60 | A |
| 102 | 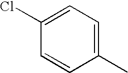 | 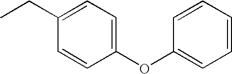 | H | —CH₃ | 141 | white powder | 51 | A |
| 103 | 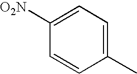 | 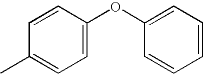 | H | —CH₃ | 148 | beige powder | 20 | A |
| 104 | 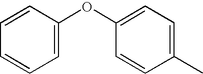 | 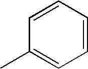 | —OH | —CH₃ | 176 | white powder | 3 | E |
| 105 | 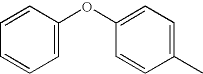 | 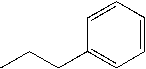 | H | —CH₃ | NMR 105 | oil | 35 | A |
| 106 | 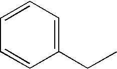 | 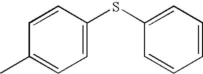 | H | —CH₃ | 146 | pale yellow crystals | 64 | A |
| 107 | 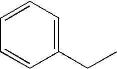 | 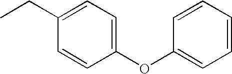 | H | —CH₃ | NMR 107 | oil | 83 | A |
| 108 | 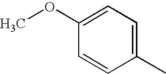 | 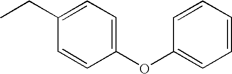 | —CH₃ | —CH₃ | 128 | white crystals | 44 | A |
| 109 | 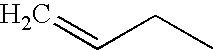 | 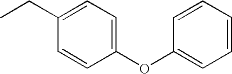 | H | —CH₃ | NMR 109 | oil | 50 | E |
| 110 | 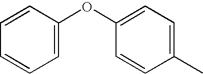 | 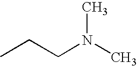 | H | —CH₃ | 168 (*) | white powder | 92 | A |
| 111 | 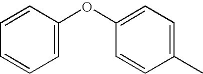 | 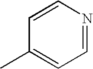 | H | —CH₃ | 234 (*) | off-white powder | 88 | A |
| 112 | 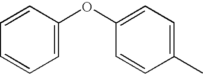 | 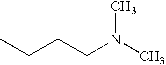 | H | —CH₃ | 205 (*) | white powder | 90 | A |
| 113 | 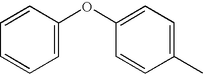 | 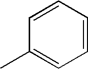 | H | 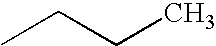 | 240 | pale yellow powder | 60 | A |
| 114 | 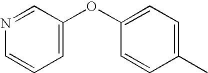 | 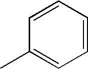 | H | —CH₃ | 189 | white powder | 55 | A |
| 115 | 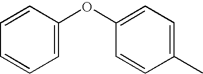 | 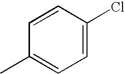 | —CH₃ | —CH₃ | 188 | white powder | 55 | A |
| 116 | 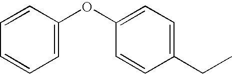 | 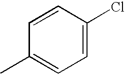 | H | —CH₃ | 60 | beige solid | 63 | E |
| 117 | 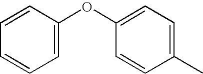 | 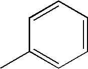 | H | 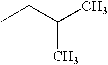 | NMR 117 | brown oil | 81 | A |
| 118 | 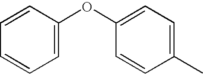 | 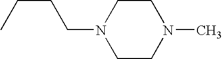 | H | —CH₃ | 243 (*) | off-white powder | 71 | A |
| 119 | 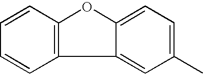 | 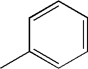 | H | —CH₃ | 195 | off-white powder | 10 | A |
| 120 | 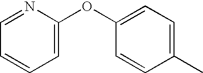 | 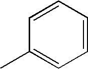 | H | —CH₃ | 236 | white powder | 39 | A |
| 121 | 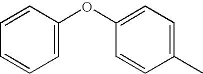 | 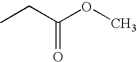 | H | —CH₃ | 50 | white foam | 63 | A |
| 122 | 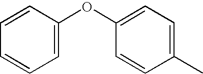 | 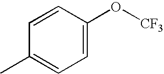 | H | —CH₃ | 128 | beige powder | 100 | A |
| 123 | 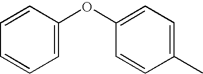 | 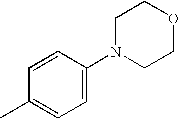 | H | —CH₃ | 154 | brown solid | 34 | A |
| 124 | 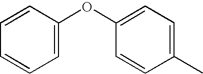 | 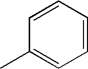 | F | F | 118 | white powder | 6 | (**) |
| 125 | 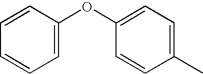 | 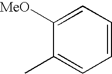 | H | —CH₃ | 192 | white crystals | 51 | A |
| 126 | 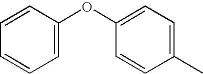 | 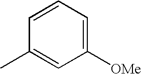 | H | —CH₃ | 175 | white crystals | 72 | A |
| 127 | 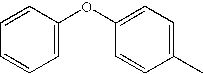 | 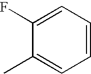 | H | —CH₃ | 76 | white crystals | 70 | A |
| 128 | 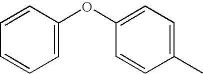 | 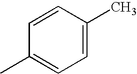 | H | —CH₃ | 183 | white crystals | 66 | A |
| 129 | 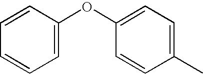 | 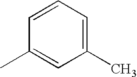 | H | —CH₃ | 160 | white crystals | 66 | A |
| 130 | 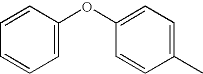 | 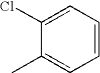 | H | —CH₃ | 165 | white crystals | 42 | A |
| 131 | 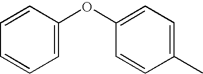 | 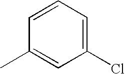 | H | —CH₃ | 155 | white crystals | 63 | A |
| 132 | 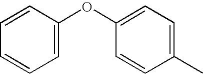 | 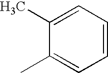 | H | —CH₃ | 155 | white crystals | 61 | A |
| 133 | 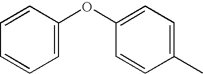 | 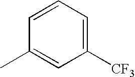 | H | —CH₃ | 143 | yellow powder | 74 | A |
| 134 | 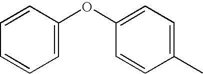 | 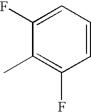 | H | —CH₃ | 130 | yellow solid | 80 | A |
| 135 | 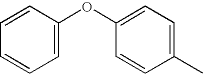 | 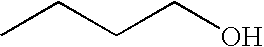 | H | —CH₃ | NMR 135 | gum | 8.5 | (***) |
| 136 | 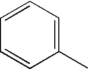 | 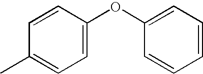 | H | —C₂H₅ | 138 | white crystals | 32 | A |
| 137 | 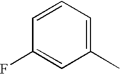 | 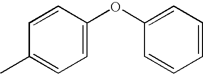 | H | —C₂H₅ | 114 | white crystals | 73 | E |

(*) hydrochloride
(**) Ex. 124: This compound is prepared on the basis of Example 31, in carbon tetrachloride, by reacting N-fluorobenzenesulfonimide (2 equivalents) and DAST (diethylaminosulfur trifluoride, 3 equivalents) at the reflux temperature of the solvent for 10 h, and then purifying the crude product by chromatography on silica gel.
(***) Ex. 135: This compound is prepared in dichloromethane by reacting thiocarbonyldiimidazole with 3-aminopropanol, in the presence of the amino acid obtained according to Preparation I and triethylamine, for 24 h at room temperature, and then purifying the crude product by chromatography on silica gel.

NMR 60

¹H NMR (DMSO d₆, 300 MHz): 1.3 (d, 3H); 4.37 (m, 2H); 4.70 (q, 1H); 5.11 (m, 2H); 5.81 (m, 1H); 6.96 (d, 1H); 7.05 (d, 2H); 7.18 (t, 1H); 7.25 (t, 1H); 7.38 (m, 3H); 7.53 (d, 1H).

NMR 79

¹H NMR (DMSO d₆, 250 MHz): 4.80 (s, 2H); 4.99 (s, 2H); 6.98 (m, 4H); 7.14 (t, 1H); 7.39 (m, 7H); 7.69 (d, 2H).

NMR 80

¹H NMR (DMSO d₆, 250 MHz): 1.26 (d, 3H); 4.99 (s, 2H); 5.03 (m, 1H); 6.99 (m, 4H); 7.14 (t, 1H); 7.42 (m, 7H); 7.53 (m, 2H).

NMR 81

¹H NMR (DMSO d₆, 300 MHz): 1.37 (d, 6H); 5.02 (s, 2H); 7.00 (t, 4H); 7.15 (t, 1H); 7.38 (m, 6H); 7.53 (m, 3H).

NMR 97

¹H NMR (DMSO d₆, 300 MHz): 1.33 (d, 3H); 4.27 (q, 1H); 4.36 (m, 2H); 4.78 (d, 1H); 5.12 (dd, 2H); 5.23 (d, 1H); 5.83 (m, 1H); 6.99 (m, 4H); 7.14 (t, 1H); 7.39 (m, 4H).

NMR 99

¹H NMR (DMSO d₆, 300 MHz): 1.27 (d, 3H); 4.39 (d, 2H); 5.00 (q, 1H); 5.17 (m, 2H); 5.83 (m, 1H); 7.06 (m, 3H); 7.18 (t, 1H); 7.28 (m, 2H); 7.45 (m, 3H).

NMR 105

¹H NMR (DMSO d₆, 300 MHz): 1.17 (d, 3H); 2.97 (m, 2H); 3.98 (t, 2H); 4.83 (q, 1H); 7.08 (d, 4H); 7.31 (m, 6H); 7.46 (m, 4H).

NMR 107

¹H NMR (DMSO d₆, 250 MHz): 1.30 (d, 3H); 4.32 (q, 1H); 4.84 (d, 1H); 4.94 (s, 2H); 5.21 (d, 1H); 6.98 (t, 4H); 7.17 (m, 1H); 7.32 (m, 9H).

NMR 109

¹H NMR (DMSO d₆, 300 MHz): 1.37 (d, 3H); 4.18 (dd, 1H); 4.39 (q, 1H); 4.59 (dd, 1H); 4.90 (s, 2H); 5.27 (dd, 2H); 5.81 (m, 1H); 6.97 (m, 4H); 7.11 (t, 1H); 7.36 (m, 4H).

NMR 117

¹H NMR (CDCl₃, 250 MHz): 0.9 (dd, 6H); 1.83 (m, 3H); 4.66 (q, 1H); 7.09 (m, 4H); 7.12 (t, 1H); 7.38 (m, 6H); 7.50 (m, 3H).

NMR 135

¹H NMR (CDCl₃, 250 MHz): 1.43 (d, 3H); 1.96 (m, 2H); 2.67 (t, 1H); 3.63 (q, 2H); 4.10 (t, 2H); 4.43 (q, 1H); 7.07 (m, 4H); 7.18 (t, 1H); 7.29 (m, 2H); 7.38 (m, 2H).

Examples 138 to 148 below illustrate the compounds of formula (I) in which A is —CH₂—O— or —O—CH₂—.

### EXAMPLE 138

### 5-Methyl-1-[4-(phenylmethoxy)phenyl]-3-(2-propenyl)-2-thioxoimidazolidin-4-one

A mixture of 0.6 g (2.2 mmol) of the acid obtained according to Preparation XXVI and 18 ml of acetonitrile is prepared. 0.5 ml (3.7 ml) of triethylamine is added (giving a solution), followed by 0.325 ml (3.3 mmol) of allyl isothiocyanate. The reaction mixture is stirred for 15 h at room temperature and the solvent is then removed under reduced pressure. The residue is purified by chromatography on silica gel using a cyclohexane/ethyl acetate mixture (9/1; v/v) as the eluent to give 0.73 g of the expected product in the form of a white solid (yield=93%).

M.p.=88-90° C.

### EXAMPLE 139

### 3-(4-Methoxyphenyl)-5-methyl-1-[4-(phenylmethoxy)phenyl-2-thioxoimidazolidin-4-one

A procedure analogous to that of Example 138 is followed, except that the allyl isothiocyanate is replaced by 4-methoxyphenyl isothiocyanate, to give the expected compound in the form of white crystals (yield=48%).

M.p.=182-184° C.

### EXAMPLE 140

### 3-(4-Chlorophenyl)-5-methyl-1-[4-(phenylmethoxy)phenyl]-2-thioxoimidazolidin-4-one

A procedure analogous to that of Example 138 is followed, except that the allyl isothiocyanate is replaced by 4-chlorophenyl isothiocyanate, to give the expected product in the form of a white powder (yield=47%).

M.p.=180-183° C.

### EXAMPLE 141

### 5-Methyl-3-phenyl-1-[4-(phenylmethoxy)phenyl]-2-thioxoimidazolidin-4-one

A solution of 2 g (6.7 mmol) of the ester obtained according to Preparation XXV in 20 ml of xylene is prepared and 1 g (7.4 mmol) of phenyl isothiocyanate and 6.6 ml of acetic acid are added. The reaction mixture is heated for 2 h at 110° C., with stirring, and then concentrated under reduced pressure. The residue is purified by chromatography on silica gel using dichloromethane as the eluent. The pure fraction is crystallized from ethyl ether, filtered off and dried to give 0.67 g of the expected product in the form of orange-yellow crystals (yield=26%).

M.p.=152-154° C.

### EXAMPLE 142

### 5-Methyl-1-phenyl-3-[4-(phenylmethoxy)phenyl]-2-thioxoimidazolidin-4-one

A mixture of 77.5 g (0.50 mol) of N-phenylalanine and 76.5 ml of triethylamine in 1.45 l of ethanol is prepared. The solution obtained is filtered on a glass frit, 133 g (0.55 mol) of 4-(phenylmethoxy)phenyl isothiocyanate are then added and the reaction mixture is stirred at room temperature for 18 hours. The precipitate formed is filtered off and then dissolved in an ethanol/dichloromethane mixture. The solution obtained is treated with active charcoal, filtered and partially concentrated under reduced pressure. The white precipitate formed is filtered off, washed with ethanol and dried under vacuum to give the expected compound in the form of white crystals (yield=52%).

M.p.=155° C.

### EXAMPLE 143

### 1-(4-Methoxyphenyl)-5-methyl-3-[4-(phenylmethoxy)phenyl]-2-thioxoimidazolidin-4-one

A procedure analogous to that of Example 142 is followed, except that N-(4-methoxyphenyl)alanine and 4-(phenylmethoxy)phenyl isothiocyanate are used as the starting materials, to give the expected product in the form of white crystals (yield=95%).

M.p.=184-186° C.

### EXAMPLE 144

### 3-Phenyl-1-[4-(phenylmethoxy)phenyl]-2-thioxoimidazolidin-4-one

A procedure analogous to that of Example 141 is followed, except that the compound obtained according to Preparation XXVII is used as the starting material, to give the expected product in the form of beige crystals (yield=46%).

M.p.=194-196° C.

### EXAMPLE 145

### 5,5-Dimethyl-3-phenyl-1-[4-(phenylmethoxy)phenyl]-2-thioxoimidazolidin-4-one

A procedure analogous to that of Example 142 is followed, except that the acid obtained according to Preparation XXIX is used as the starting material, to give the expected product in the form of a white powder (yield=40%).

M.p.=208-210° C.

### EXAMPLE 146

### 3-Butyl-5-methyl-1-[4-(phenylmethoxy)phenyl]-2-thioxoimidazolidin-4-one

A procedure analogous to that of Example 138 is followed, except that the compound obtained according to Preparation XXVI and butyl isothiocyanate are used as the starting materials, to give the expected product in the form of a colorless oil (yield=71.5%).

¹H NMR (300 MHz, DMSO): 7.40 (m, 7H); 7.10 (m, 2H); 5.13 (s, 2H); 4.82 (q, 1H); 3.77 (t, 2H); 1.61 (q, 2H); 1.29 (q, 2H); 1.21 (d, 3H); 0.91 (t, 3H).

### EXAMPLE 147

### 5-Methyl-1-phenylmethyl-3-[4-(phenylmethoxy)phenyl]-2-thioxoimidazolidin-4-one

A procedure analogous to that of Example 138 is followed, except that 4-(phenylmethoxy)phenyl isothiocyanate and N-(phenylmethyl)alanine are used as the starting material, to give the expected product in the form of pale yellow crystals (yield=60%).

M.p.=156° C.

### EXAMPLE 148

### 1-[4-(Phenylmethoxy)phenyl]-3-(2-propenyl)-2-thioxoimidazolidin-4-one

A procedure analogous to that of Example 141 is followed, except that the ester obtained according to Preparation XXVII and allyl isothiocyanate are used as the starting materials in toluene, to give the expected product in the form of a white powder (yield=20%).

M.p.=130° C.

Table III collates the compounds described in Examples 138 to 148:

TABLE III

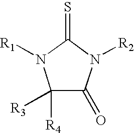

| Ex. | R₁ | R₂ | R₃ | R₄ |
|---|---|---|---|---|
| 138 | 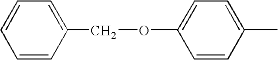 | —CH₂—CH═CH₂ | CH₃ | H |
| 139 | 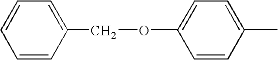 | 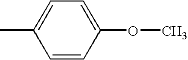 | CH₃ | H |
| 140 | 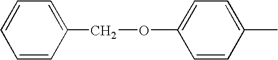 | 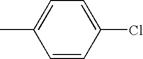 | CH₃ | H |
| 141 | 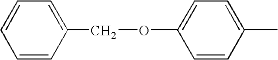 | 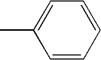 | CH₃ | H |
| 142 | 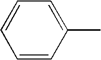 | 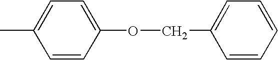 | CH₃ | H |
| 143 | 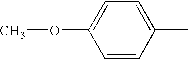 | 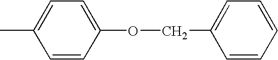 | CH₃ | H |
| 144 | 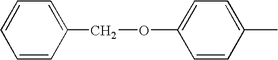 | 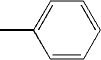 | H | H |
| 145 | 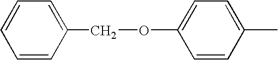 | 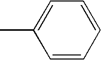 | CH₃ | CH₃ |
| 146 | 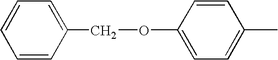 | —(CH₂)₃—CH₃ | CH₃ | H |
| 147 | 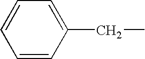 | 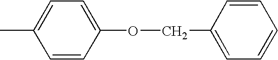 | CH₃ | H |
| 148 | 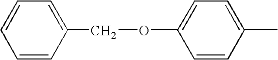 | —CH₂—CH═CH₂ | H | H |

The compounds according to Examples 149 to 184, collated in Table IV, were obtained by preparative methods analogous to those used for Examples 138 to 148 described above. The melting point (M.p.) in ° C., the appearance, the yield of the synthesis (Y) and the preparative method used (A: from an acid, analogously to Example 142; E: from an ester, analogously to Example 141) are indicated for each of these Examples.

TABLE IV

| | | | | | | Appear- | | |
|---|---|---|---|---|---|---|---|---|
| Example | R₁ | R₂ | R₃ | R₄ | M.p. | ance | Yield | Method |
| 149 | 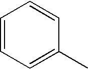 | 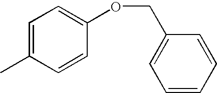 | H | H | 217 | pale yellow powder | 48 | B |
| 150 | 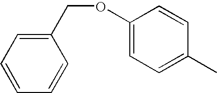 | 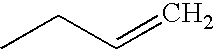 | 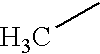 | 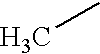 | 130 | off- white powder | 58 | A |
| 151 | 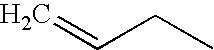 | 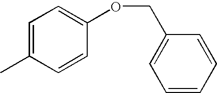 | H | 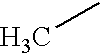 | 127 | off- white powder | 90 | E |
| 152 | 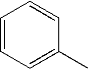 | 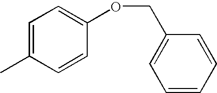 | 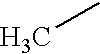 | 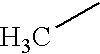 | 208 | white powder | 20 | E |
| 153 | 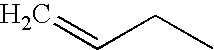 | 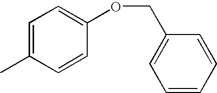 | H | H | 162 | pale yellow powder | 54 | E |
| 154 | 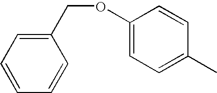 | 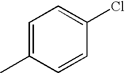 | 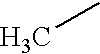 | 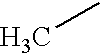 | 176 | white powder | 62 | A |
| 155 | 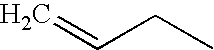 | 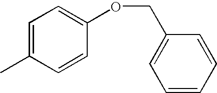 | 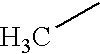 | 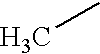 | 133 | white powder | 29 | E |
| 156 | 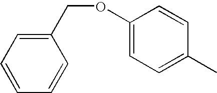 | 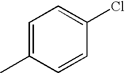 | H | H | 196 | white powder | 69 | A |
| 157 | 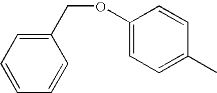 | 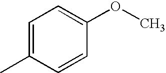 | 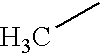 | 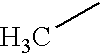 | 225 | white powder | 40 | A |
| 158 | 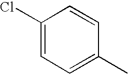 | 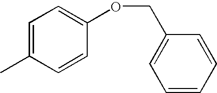 | H | 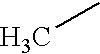 | 196 | off- white powder | 40 | A |
| 159 | 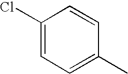 | 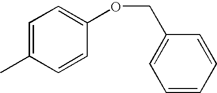 | H | H | 196 | pale yellow powder | 24 | A |
| 160 | 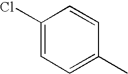 | 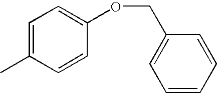 | 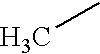 | 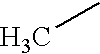 | 195 | white powder | 32 | A |
| 161 | 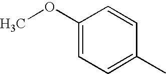 | 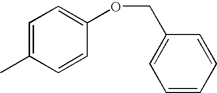 | 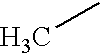 | 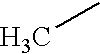 | 216 | white powder | 28 | A |
| 162 | 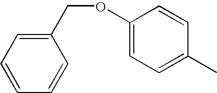 | 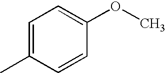 | H | H | 210 | off- white cotton | 48 | A |
| 163 | 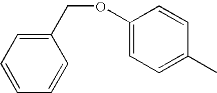 | 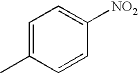 | H | 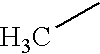 | 216 | white powder | 69 | A |
| 164 | 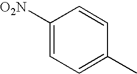 | 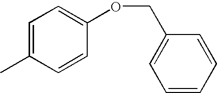 | H | 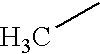 | 171 | yellow powder | 7 | A |
| 165 | 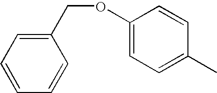 | 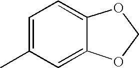 | H | 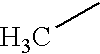 | 154 | off- white powder | 84 | A |
| 166 | 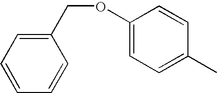 | 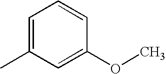 | H | 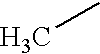 | 120 | white powder | 72 | A |
| 167 | 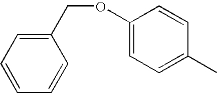 | 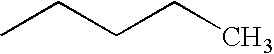 | H | H | 118 | pale yellow powder | 51 | E |
| 168 | 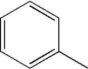 | 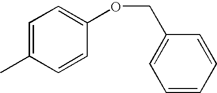 | H | 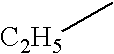 | 126 | cream- colored crystals | 61 | A |
| 169 | 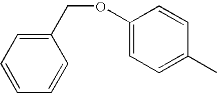 | 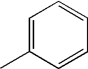 | H | 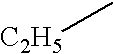 | 172 | cristaux blancs | 72 | E |
| 170 | 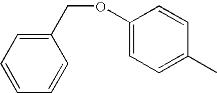 | 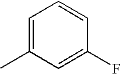 | H | 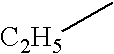 | 171 | white crystals | 83 | E |
| 171 | 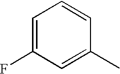 | 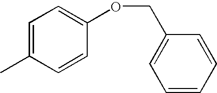 | H | 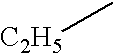 | 143 | white crystals | 36 | E |
| 172 | 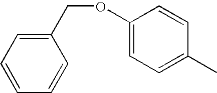 | 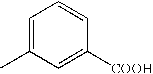 | H | 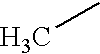 | 247 | beige solid | 40 | E |
| 173 | 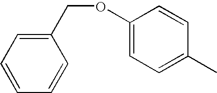 | 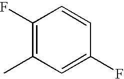 | H | 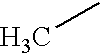 | 163 | white solid | 72 | E |
| 174 | 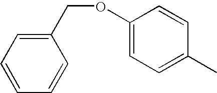 | 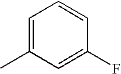 | H | 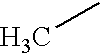 | 157 | white solid | 68 | E |
| 175 | 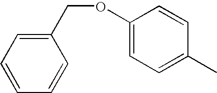 | 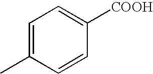 | H | 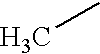 | 260 | beige solid | 52 | E |
| 176 | 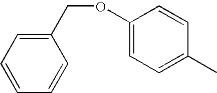 | 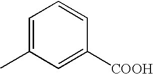 | 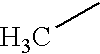 | 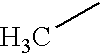 | 208 | white powder | 51 | E |
| 177 | 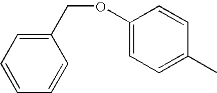 | 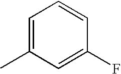 | 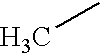 | 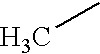 | 182 | white powder | 50 | E |
| 178 | 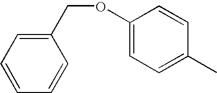 | 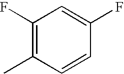 | 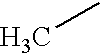 | 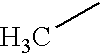 | 169 | white powder | 24 | E |
| 179 | 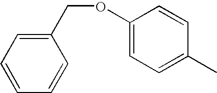 | 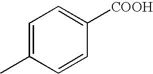 | 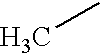 | 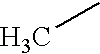 | 250 | white crystals | 22 | E |
| 180 | 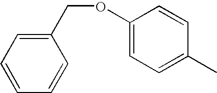 | 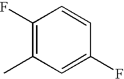 | 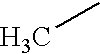 | 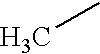 | 160 | white powder | 45 | E |
| 181 | 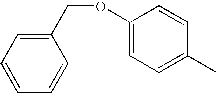 | 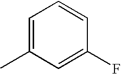 | H | 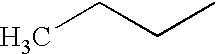 | 124 | white powder | 41 | E |
| 182 | 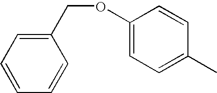 | 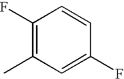 | H | 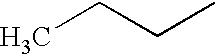 | 140 | white powder | 75 | E |
| 183 | 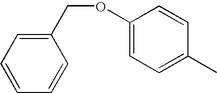 | 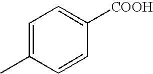 | H | 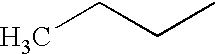 | 220 | white powder | 82 | E |
| 184 | 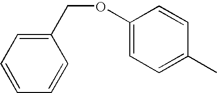 | 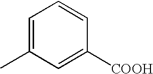 | H | 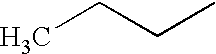 | 199 | white powder | 47 | E |

The Examples which follow relate to compounds of formula (I) according to the invention in which A is the —CH₂— group:

### EXAMPLE 185

### 5-Methyl-1-phenyl-3-[4-(phenylmethyl)phenyl]-2-thioxoimidazolidin-4-one

A mixture of 165 g (1 mol) of N-phenylalanine and 153 ml of triethylamine in 2 l of ethanol is prepared. The solution obtained is filtered on a glass frit and 247.5 g (1.1 mol) of the compound obtained according to Preparation XXXIII are added. The mixture is stirred for 18 hours at room temperature. The precipitate obtained is filtered off and then dissolved in a dichloromethane/ethanol mixture. The solution is treated with active charcoal and then filtered and partially concentrated on a rotary evaporator. The product which has precipitated is filtered off, washed with ethanol and dried to give the expected product with a yield of 36%.

M.p.=123-125° C.

### EXAMPLE 186

### 5-Methyl-3-(2-propenyl)-1-[4-(phenylmethyl)phenyl]-2-thioxoimidazolidin-4-one

A solution of 0.2 g (0.71 mmol) of the ester obtained according to Preparation XXXIV in 3 ml of toluene is prepared and 0.6 ml of acetic acid and 0.07 g (0.71 mmol) of allyl isothiocyanate are added. The reaction mixture is maintained at the reflux temperature of the solvent for 5 h, with stirring, and then concentrated under reduced pressure. The residue is purified by chromatography on silica gel using a cyclohexane/ethyl acetate mixture (95/5; v/v) as the eluent to give 160 mg of the expected product in the form of a light yellow solid (yield=67%).

M.p.=62-64° C.

### EXAMPLE 187

### 5-Methyl-3-phenyl-1-[4-(phenylmethyl)phenyl]-2-thioxoimidazolidin-4-one

A procedure analogous to that of Example 185 is followed, except that the acid obtained according to Preparation XXXV is used as the starting material, to give the expected product in the form of a fine and lightweight white solid (yield=21%).

M.p.=164-166° C.

### EXAMPLE 188

### 5-Methyl-3-(4-nitrophenyl)-1-[4-(phenylmethyl)phenyl]-2-thioxoimidazolidin-4-one

A mixture of 0.51 g (2 mmol) of the acid obtained according to Preparation XXXV and 15 ml of acetonitrile is prepared. 0.45 ml (3.3 mmol) of triethylamine is added, followed by 0.55 g (3 mmol) of 4-nitrophenyl isothiocyanate. The reaction mixture is stirred for 15 hours at room temperature and then concentrated under reduced pressure. The residue is purified by chromatography on silica gel using a cyclohexane/ethyl acetate mixture (9/1; v/v) as the eluent to give the expected product in the form of a yellow powder (yield=46%).

M.p.=200° C.

### EXAMPLE 189

### 3-(4-Chlorophenyl)-5-methyl-1-[4-(phenylmethyl)phenyl]-2-thioxoimidazolidin-4-one

A procedure analogous to that of Example 188 is followed, except that 4-chlorophenyl isothiocyanate is used as the starting material, to give the expected product in the form of a yellow solid (yield=51%).

M.p.=144° C.

### EXAMPLE 190

### 3-[4-(Phenylmethyl)phenyl]-1-(2-propenyl)-2-thioxoimidazolidin-4-one

A solution of 2.40 (2 mmol) of the ethyl ester of N-alkylglycine in 25 ml of toluene is prepared and 0.5 g (2.2 mmol) of the isothiocyanate obtained according to Preparation XXXIII and 2.2 ml of acetic acid are added. The reaction mixture is heated gently at the reflux temperature of the solvent for 2 hours, with stirring, and then concentrated under reduced pressure. The residue is purified by chromatography on silica gel using dichloromethane as the eluent to give the expected product in the form of a beige powder (yield=65%).

M.p.=108° C.

### EXAMPLE 191

### 3-Phenyl-1-[4-(phenylmethyl)phenyl]-2-thioxoimidazolidin-4-one

A procedure analogous to that of Example 188 is followed, except that the acid obtained according to Preparation XXXVI and phenyl isothiocyanate are used as the starting materials in ethanol, to give the expected product in the form of cream-colored crystals (yield=20%).

M.p.=182° C.

### EXAMPLE 192

### 1-[4-(Phenylmethyl)phenyl]-3-(2-propenyl)-2-thioxoimidazolidin-4-one

A procedure analogous to that of Example 188 is followed, except that the acid obtained according to Preparation XXXVI and allyl isothiocyanate are used as the starting materials, to give the expected product in the form of a white powder (yield=85%).

M.p.=132° C.

### EXAMPLE 193

### 3-(4-Nitrophenyl)-1-[4-(phenylmethyl)phenyl]-2-thioxoimidazolidin-4-one

A procedure analogous to that of Example 188 is followed, except that the acid obtained according to Preparation XXXVI and 4-nitrophenyl isothiocyanate are used as the starting materials, to give the expected product in the form of a pale yellow powder (yield=30%).

M.p.=209° C.

### EXAMPLE 194

### 3-Phenylmethyl-1-[4-(phenylmethyl)phenyl]-2-thioxoimidazolidin-4-one

A procedure analogous to that of Example 193 is followed, except that benzyl isothiocyanate is used as the starting material, to give the expected product in the form of white crystals (yield=57%).

M.p.=107° C.

### EXAMPLE 195

### 3-(4-Methoxyphenyl)-1-[4-(phenylmethyl)phenyl]-2-thioxoimidazolidin-4-one

A procedure analogous to that of Example 193 is followed, except that 4-methoxyphenyl isothiocyanate is used as the starting material, to give the expected product in the form orange flakes (yield=65%).

M.p.=164° C.

### EXAMPLE 196

### 1-[4-(Phenylmethyl)phenyl]-3-(2-propenyl)-2-thioxoimidazolidin-4-one

A procedure analogous to that of Example 193 is followed, except that 2-propenyl isothiocyanate is used as the starting material, to give the expected product in the form of a white powder (yield=27%).

M.p.=162° C.

### EXAMPLE 197

### 1-Phenyl-3-[4-(phenylmethyl)phenyl]-2-thioxoimidazolidin-4-one

A procedure analogous to that of Example 190 is followed, except that the ethyl ester of N-phenylglycine is used as the starting material, to give the expected product in the form of a pale yellow powder (yield=67%).

M.p.=194° C.

### EXAMPLE 198

### 1-(4-Chlorophenyl)-3-[4-(phenylmethyl)phenyl]-2-thioxoimidazolidin-4-one

A procedure analogous to that of Example 197 is followed, except that the ethyl ester of N-(4-chlorophenyl)glycine is used as the starting material, to give the expected product in the form of orange crystals (yield=63%).

M.p.=153° C.

### EXAMPLE 199

### 5-Methyl-3′-[4-(phenylmethyl)phenyl]-1-(2-propenyl)-2-thioxoimidazolidin-4-one

A procedure analogous to that of Example 190 is followed, except that 4-(phenylmethyl)phenyl isothiocyanate is used as the starting material, to give the expected product in the form of a lightweight white powder (yield=78%).

M.p.=135° C.

### EXAMPLE 200

### 3-(4-Methoxyphenyl)-5-methyl-1-[4-(phenylmethyl)phenyl]-2-thioxoimidazolidin-4-one

A procedure analogous to that of Example 186 is followed, except that 4-methoxyphenyl isothiocyanate is used as the starting material, to give the expected product in the form of white crystals (yield=75%).

M.p.=122° C.

### EXAMPLE 201

### 5,5-Dimethyl-3-(4-methoxyphenyl)-1-[4-(phenylmethyl)phenyl]-2-thioxoimidazolidin-4-one

A procedure analogous to that of Example 185 is followed, except that the acid obtained according to Preparation XXXVII and 4-methoxyphenyl isothiocyanate are used as the starting materials, to give the expected product in the form of white crystals (yield=27%).

M.p.=162° C.

The chemical structures of compounds 185 to 201 described above are summarized in Table V below.

Table VI collates other compounds according to the invention, obtained by preparative methods analogous to those described for Examples 185 to 201; the melting points (M.p. ° C.), the yields of the preparation and the synthetic method used (A analogously to Example 185; E analogously to Example 186) are indicated in this Table.

TABLE V

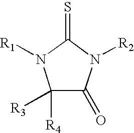

| Ex. | R₁ | R₂ | R₃ | R₄ |
|---|---|---|---|---|
| 185 | 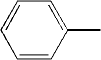 | 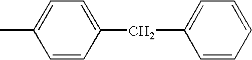 | CH₃ | H |
| 186 | 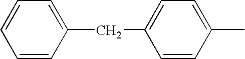 | —CH₂—CH═CH₂ | CH₃ | H |
| 187 | 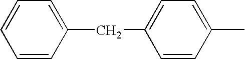 | 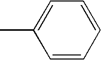 | CH₃ | H |
| 188 | 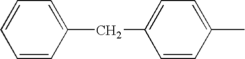 | 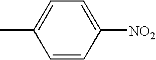 | CH₃ | H |
| 189 | 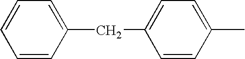 | 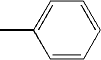 | H | H |
| 190 | CH₂═CH—CH₂— | 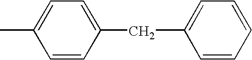 | H | H |
| 191 | 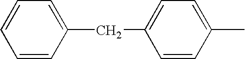 | 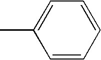 | H | H |
| 192 | 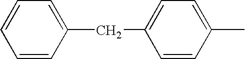 | —CH₂—CH═CH₂ | H | H |
| 193 | 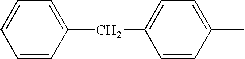 | 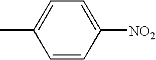 | H | H |
| 194 | 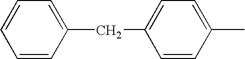 | 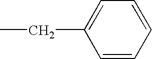 | H | H |
| 195 | 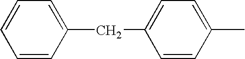 | 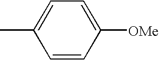 | H | H |
| 196 | 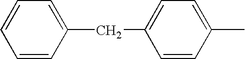 | —CH₂—CH═CH₂ | H | H |
| 197 | 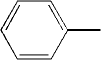 | 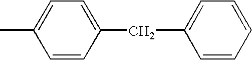 | H | H |
| 198 | 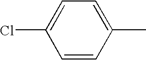 | 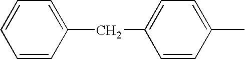 | H | H |
| 199 | CH₂═CH—CH₂— | 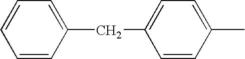 | CH₃ | H |
| 200 | 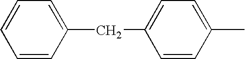 | 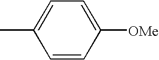 | CH₃ | H |
| 201 | 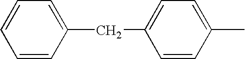 | 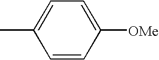 | CH₃ | CH₃ |

TABLE VI

| | | | | | M.p. | Appear- | | |
|---|---|---|---|---|---|---|---|---|
| Ex. | R₁ | R₂ | R₃ | R₄ | ° C. | ance | Yield | Meth. |
| 202 | 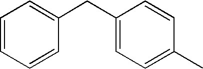 | 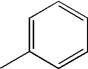 | 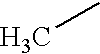 | 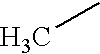 | 184 | white powder | 56 | A |
| 203 | 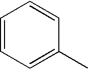 | 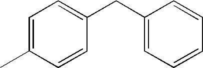 | H | H | 232 | white powder | 89 | A |
| 204 | 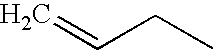 | 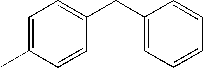 | 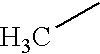 | 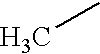 | 98 | white powder | 95 | E |
| 205 | 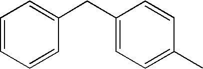 | 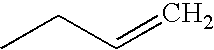 | 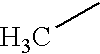 | 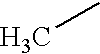 | NMR | white solid | 29 | A |
| 206 | 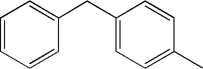 | 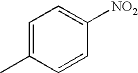 | 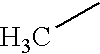 | 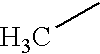 | 185 | yellow powder | 62 | A |
| 207 | 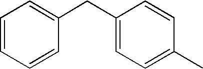 | 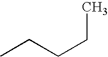 | H | H | 78 | white crystals | 58 | A |
| 208 | 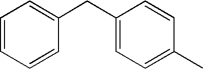 | 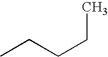 | H | 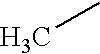 | NMR | pale yellow oil | 67 | A |
| 209 | 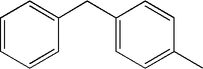 | 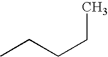 | 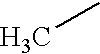 | 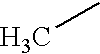 | NMR | pale yellow oil | 52 | A |
| 210 | 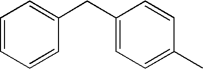 | 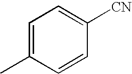 | 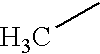 | 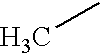 | 188 | white cotton | 66 | A |
| 211 | 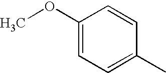 | 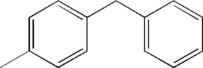 | 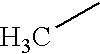 | 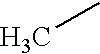 | 233 | white powder | 40 | A |
| 212 | 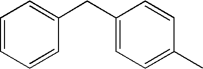 | 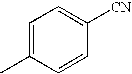 | H | 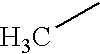 | 154 | off- white powder | 52 | A |
| 213 | 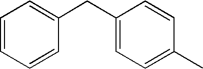 | 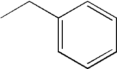 | 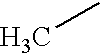 | 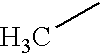 | 130 | white powder | 58 | A |
| 214 | 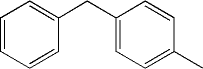 | 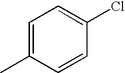 | H | H | 186 | pale yellow powder | 49 | A |
| 215 | 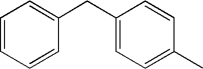 | 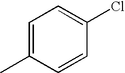 | H | H | 160 | beige solid | 71 | A |
| 216 | 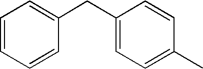 | 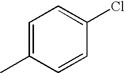 | 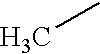 | 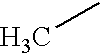 | 167 | white powder | 73 | A |
| 217 | 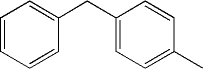 | 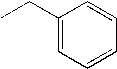 | H | 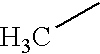 | 50 | green crystals | 95 | A |
| 218 | 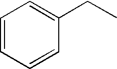 | 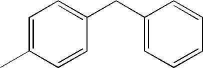 | H | H | 153 | white powder | 82 | A |
| 219 | 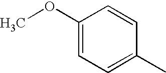 | 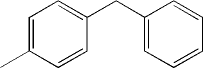 | H | 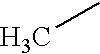 | 202 | white crystals | 76 | A |
| 220 | 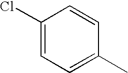 | 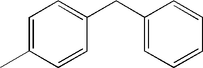 | H | 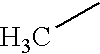 | 140 | white powder | 21 | A |
| 221 | 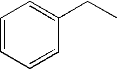 | 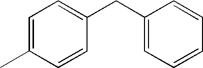 | 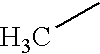 | 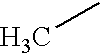 | 129 | white powder | 79 | A |
| 222 | 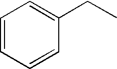 | 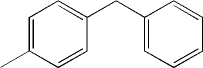 | H | 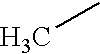 | 118 | white powder | 57 | A |
| 223 | 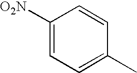 | 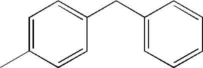 | H | 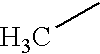 | 147 | light yellow crystals | 17 | A |
| 224 | 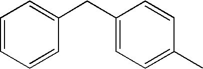 | 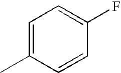 | H | H | 157 | white crystals | 56 | E |
| 225 | 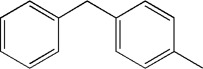 | 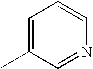 | H | H | 185 | light yellow crystals | 80 | E |
| 226 | 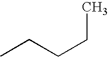 | 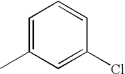 | H | H | 142 | pale orange crystals | 75 | E |
| 227 | 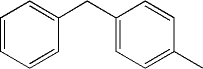 | 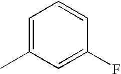 | H | H | 172 | white crystals | 72 | E |
| 228 | 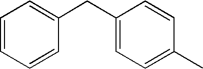 | 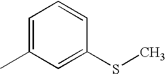 | H | H | 130 | light beige crystals | 76 | E |
| 229 | 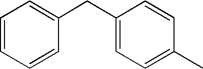 | 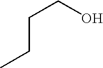 | H | H | 88 | light orange crystals | 35 | (*) |
| 230 | 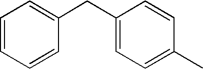 | 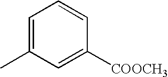 | H | H | 146 | orange flakes | 41 | E |
| 231 | 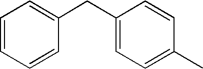 | 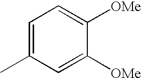 | H | H | 160 | cream- colored crystals | 65 | E |
| 232 | 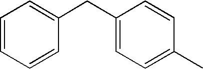 | 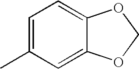 | H | H | 205 | beige crystals | 71 | E |
| 233 | 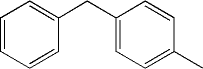 | 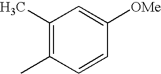 | H | H | 132 | pale pink crystals | 63 | E |
| 234 | 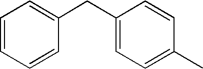 | 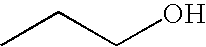 | H | H | 132 | white crystals | 69 | (**) |
| 235 | 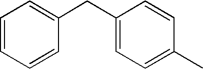 | 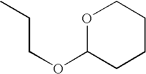 | H | H | NMR | gum | 100 | (***) |
| 236 | 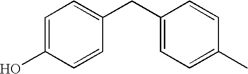 | 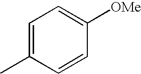 | H | H | 174 | white powder | 60 | E |
| 237 | 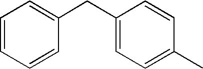 | 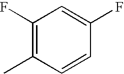 | H | H | 135 | light red crystals | 89 | E |
| 238 | 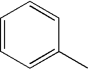 | 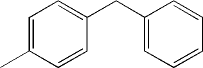 | H | 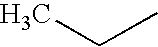 | 143 | white crystals | 64 | A |
| 239 | 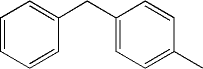 | 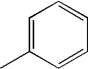 | H | 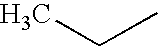 | 155 | pale orange crystals | 25 | E |
| 240 | 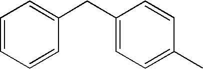 | 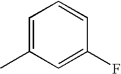 | H | 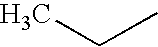 | 60 | cream- colored foam | 79 | E |
| 241 | 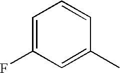 | 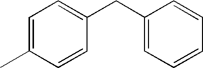 | H | 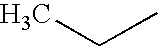 | 150 | white crystals | 27 | E |
| 242 | 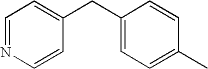 | 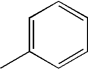 | H | 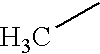 | 220 | beige powder | 27 | A |
| 243 | 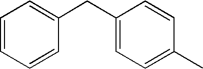 | 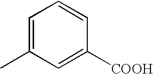 | H | 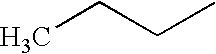 | 102 | beige solid | 22 | E |
| 244 | 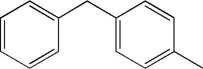 | 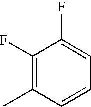 | H | 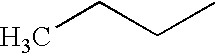 | 112 | yellow solid | 60 | E |
| 245 | 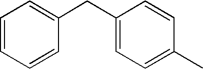 | 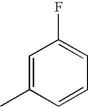 | H | 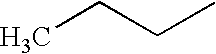 | 77 | yellow solid | 80 | E |
| 246 | 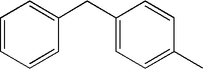 | 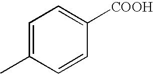 | H | 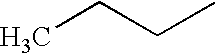 | 230 | white solid | 40 | E |
| 247 | 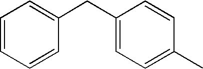 | 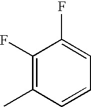 | 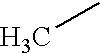 | 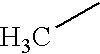 | 131 | yellow solid | 15 | E |
| 248 | 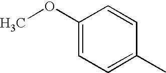 | 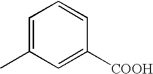 | 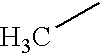 | 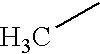 | 120 | pink solid | 45 | E |
| 249 | 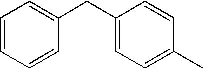 | 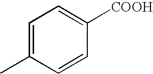 | 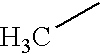 | 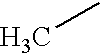 | 245 | white solid | 7 | E |

(*) Example 229: This compound is obtained by reacting 3-aminopropanol (1.1 equivalents) and 1,1′-thiocarbonyldiimidazole (1.1 equivalents) with the acid obtained according to Preparation XXVI in a dichloromethane/methanol mixture for 1 hour at 45° C.
(**) Example 234: This compound is obtained by hydrolyzing the compound of Example 235 with paratoluenesulfonic acid (0.05 equivalent) in methanol at 45° C. for 2 hours.
(***) Example 235: This compound is obtained by a process analogous to that of Example 229, starting from 2-[(tetrahydro-2H-pyran-2-yl)oxy]ethanamine.

NMR:

### EXAMPLE 205

¹H NMR (DMSO d₆, 300 MHz): 1.33 (s, 6H); 4.01 (s, 2H); 4.42 (2d, 2H); 5.15 (m, 2H); 5.87 (m, 1H); 7.30 (m, 9H).

### EXAMPLE 208

¹H NMR (CDCl₃, 300 MHz): 0.97 (t, 3H); 1.40 (m, 5H); 1.71 (m, 2H); 3.89 (t, 2H); 4.02 (s, 2H); 4.40 (q, 1H); 7.25 (m, 9H).

### EXAMPLE 209

¹H NMR (DMSO d₆, 250 MHz): 0.9 (t, 3H); 1.3 (m, 8H); 1.61 (m, 2H); 3.79 (t, 2H); 4.01 (s, 2H); 7.28 (m, 9H).

### EXAMPLE 235

¹H NMR (CDCl₃, 250 MHz): 1.68 (m, 6H); 3.50 (m, 1H); 3.81 (m, 2H); 4.08 (m, 4H); 4.18 (m, 1H); 4.37 (s, 2H); 4.69 (m, 1H); 7.26 (m, 7H); 7.44 (d, 2H).

The compounds of formula (I) according to the invention were subjected to pharmacological tests in order to evaluate their potential to reduce the blood glycemia level.

Experimental Protocol

The in vivo studies were carried out on male C₅₇BL/KsJ-db/db mice originating from CERJ (Route des Chênes Secs—BP 5—53940 Le Genest St Isle—France).

The animals are accommodated in cages fitted with a filter lid and have free access to an irradiated standard food and to filtered drinking water. All the equipment used (cages, feeding bottles, pipettes and shavings) is sterilized by autoclaving, irradiation or immersion in a disinfectant. The temperature of the room is maintained at 23±2° C. The light-dark cycle is 12 h.

During the acclimatization period, each animal is tagged with an electronic chip, which is implanted under anesthesia effected by the inhalation of a CO₂/O₂ mixture.

Groups of 8 to 10 mice are formed and the treatments start when the animals are 9 to 11 weeks old. The products are suspended in gum arabic at a concentration of 3% and administered to the animals by means of a gavage cannula for 10 days at a rate of two administrations per day, as well as on the morning of day 11. The products are tested at doses below 200 mg/kg and generally of 10 mg/kg. The animals in the control group receive the dosage vehicle only.

A blood sample is taken before treatment and then four hours after the last administration of the product. The animals are anesthetized by the inhalation of a CO₂/O₂ mixture and the blood is taken from the retro-orbital sinus, collected in a dry tube and kept cold. The serum is separated off by centrifugation at 2800 g (15 minutes, 4° C.) during the hour following sampling. The samples are kept at −20° C. until they are analyzed.

The serum glucose and triglyceride levels are determined on a Konélab 30 analyzer by means of Konélab kits. The animals whose glycemia before treatment was below 3 g/l are systematically excluded from the study.

For each group, the mean glucose and triglyceride levels after treatment are calculated and the results are expressed as the percentage variation of these means relative to the control group after verification of the homogeneity of the means before treatment.

In general terms, the experiments performed with the compounds described in the invention show very substantial decreases in glycemia and triglyceridemia, with values ranging up to −63% for glycemia and −60% for triglycerides. It was also observed that the treatment with the compounds according to the invention was accompanied by a favorable modification of the lipid parameters.

By way of example, when carrying out the pharmacological tests in accordance with the above descriptions, the results collated in Table A were observed (Gly indicates the decrease in the glycemia level and TG indicates the decrease in the triglyceride level, both expressed as percentages):

TABLE A

| Example | Gly | TG |
|---|---|---|
| 20 | −47 | −47 |
| 30 | −54 | −53 |
| 62 | −38 | −46 |
| 98 | −60 | −36 |
| 139 | −36 | −32 |
| 162 | −36 | −25 |
| 185 | −57 | −39 |
| 190 | −58 | −53 |
| 195 | −53 | −46 |
| 200 | −63 | −58 |
| 219 | −52 | −20 |

The compounds according to the invention can be used as active principles in a drug for the treatment of diabetes in mammals and, more particularly, in man. They can be used to combat hypertriglyceridemia and diseases caused by an excess of triglycerides in the blood, such as atherosclerosis.

In more general terms, they can be useful for the prevention or treatment of diseases associated with hyperglycemia or hypertriglyceridemia, such as type II diabetes, hypertension, dyslipidemia, cardiovascular diseases and obesity; they are also useful for the treatment of diseases due to microvascular or macrovascular complications in diabetics, especially in the renal system or central nervous system, said complications generally being associated with metabolic syndrome X. The compounds according to the invention are also useful for treating cerebral ischemia or cerebral vascular accident.

Pharmaceutical compositions incorporating the compounds according to the invention can be formulated in particular by combining these compounds with customary non-toxic excipients by means of processes well known to those skilled in the art, preferably to give drugs for oral administration, for example gelatin capsules or tablets. In practical terms, in the case of oral administration of the compound, the daily dosage for humans will preferably be between 5 and 500 mg. Although gelatin capsule or tablet formulations are preferred for reasons of patient comfort, the compounds according to the invention can also be prescribed in other galenical forms, for example if the patient does not accept or is not in a condition to accept solid oral formulations, or if the treatment requires a very rapid bioavailability of the active principle. Thus it will be possible to present the drug in the form of a syrup to be taken orally, or in injectable form, preferably for subcutaneous or intramuscular injection.

1. Compound derived from 2-thiohydantoin, which is selected from:
a) compounds of the formula 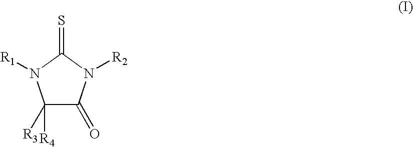
in which
R₁ or R₂ each independently is
a linear, branched or cyclic C₁-C₅ alkyl group,
a C₃-C₄ alkenyl group,
a C₂-C₃ hydroxyalkyl group or one of its precursor groups,
a C₃-C₅ alkoxyalkyl group,
a CH₂—COOCH₃ group,
an N,N-dialkylaminoalkyl group,
a group 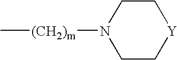
in which m is 2 or 3 and Y is O or N—CH₃,
a dibenzofuranyl group, or
a group (CH₂)ₚ-Ar, in which
p is 0 or 1, and
Ar is a phenyl or pyridinyl aromatic ring that is unsubstituted or substituted by one or more atoms or groups of atoms selected from halogens, C₁-C₄ alkyl, hydroxyl, nitro, C₁-C₃ alkoxy, methylenedioxy, SCH₃, free or esterified carboxylic acid, trifluoromethyl, trifluoromethoxy, cyano, morpholinyl and 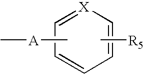
in which
A is O, S, CH₂, OCH₂ or CH₂O,
X is CH or N, and
R₅ is a hydrogen atom, a halogen atom, an N,N-dialkylamino group, a C₁-C₄ alkyl group, a C₁-C₃ alkoxy group, a hydroxyl group that is free or esterified by an amino acid, or a carboxyl or alkoxy(C₁-C₄)carbonyl group;
R₃ is a hydrogen atom, a halogen atom, a C₁-C₄ alkyl group, a C₁-C₄ alkoxy group, a hydroxyl group, a phenyl group or a benzyl group; and
R₄ is a hydrogen atom, a halogen atom or a C₁-C₄ alkyl group,
with the proviso that at least one of the substituents R₁ and R₂ comprises in its structure 2 aromatic rings selected from phenyl and pyridinyl groups, the dibenzofuranyl group being considered here as comprising 2 aromatic rings; and
b) addition salts of the compounds of formula (I) with a non-toxic acid if said compounds of formula (I) comprise a salifiable basic group.

2. Compound derived from 2-thiohydantoin, which is selected from:
a) compounds of the formula 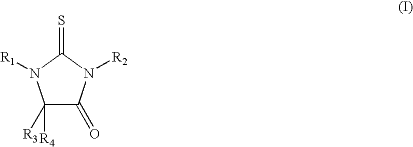
in which
R₁ and R₂ independently of one another are
a C₁-C₅ alkyl group,
a C₃-C₄ alkenyl group,
a C₂-C₃ hydroxyalkyl group,
a C₃-C₅ alkoxyalkyl group,
a CH₂—COOCH₃ group,
an N,N-dialkylaminoalkyl group,
a group 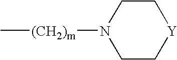
in which m is 2 or 3 and Y is O or N—CH₃,
a dibenzofuranyl group, or
a group (CH₂)ₚ-Ar in which
p is 0 or 1, and
Ar is a phenyl or pyridinyl aromatic ring that is unsubstituted or substituted by one or more atoms or groups of atoms selected from halogens, C₁-C₄ alkyl, hydroxyl, nitro, C₁-C₃ alkoxy, methylenedioxy, ester, trifluoromethyl, trifluoromethoxy, cyano, morpholinyl and the group 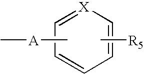
in which
A is O or S,
X is CH or N, and
R₅ is a hydrogen atom, a halogen atom, an N,N-dialkylamino group, a C₁-C₃ alkoxy group or a hydroxyl group that is free or esterified by an amino acid;
R₃ is a hydrogen atom, a halogen atom, a C₁-C₄ alkyl group, a C₁-C₄ alkoxy group, a hydroxyl group, a phenyl group or a benzyl group; and
R₄ is a hydrogen atom, a halogen atom or a C₁-C₄ alkyl group,
with the proviso that at least one of the substituents R₁ and R₂ comprises in its structure 2 aromatic rings selected from phenyl and pyridinyl groups, or is the dibenzofuranyl group; and
b) addition salts of the compounds of formula (I) with a non-toxic acid if said compounds of formula (I) comprise a salifiable basic group.

3. Compound according to claim 2, characterized in that it which is selected from:
a) compounds of the formula 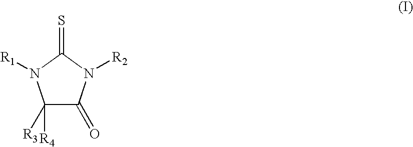
in which
R₁ is
a C₃-C₄ alkenyl group,
a dibenzofuranyl group, or
a group (CH₂)ₙ-Ar in which
n is 0 or 1, and
Ar is a phenyl or pyridinyl aromatic ring that is unsubstituted or substituted by one or more atoms or groups of atoms selected from halogens, C₁-C₄ alkyl, nitro, C₁-C₃ alkoxy, C₃-C₄ alkoxyalkyl and the group 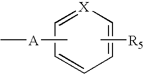
in which
A is O or S,
X is C or N, and
R₅ is a hydrogen atom, a halogen atom, an N,N-di(C₁-C₃)alkylamino group, a C₁-C₃ alkoxy group or a hydroxyl group that is free or esterified by an amino acid;
R₂ is
a C₁-C₅ alkyl group,
a C₃-C₄ alkenyl group,
a C₂-C₃ hydroxyalkyl group,
a C₃-C₅ alkoxyalkyl group,
a CH₂—COOCH₃ group,
a group N,N-di(C₁-C₃)alkylamino(C₁-C₃)alkyl,
a group 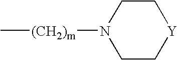
in which m is 2 or 3 and Y is O or N—CH₃, or
a group (CH₂)ₚ-Ar in which
p is 0 or 1, and
Ar is a phenyl or pyridinyl aromatic ring that is unsubstituted or substituted by one or more atoms or groups of atoms selected from halogens, C₁-C₄ alkyl, hydroxyl, nitro, C₁-C₃ alkoxy, methylenedioxy, ester, trifluoromethyl, trifluoromethoxy, cyano, morpholinyl and the group 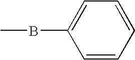
in which
B is O or S;
R₃ is a hydrogen atom, a halogen atom, a C₁-C₄ alkyl group, a C₁-C₄ alkoxy group, a hydroxyl group, a phenyl group or a benzyl group; and
R₄ is a hydrogen atom, a halogen atom or a C₁-C₄ alkyl group,
with the proviso that at least one of the substituents R₁ and R₂ comprises in its structure 2 aromatic rings selected from phenyl and pyridinyl groups, or R₁ is the dibenzofuranyl group; and
b) addition salts of the compounds of formula (I) with a non-toxic acid if said compounds of formula (I) comprise a salifiable basic group.

4. Compound derived from 2-thiohydantoin, which is selected from the compounds of formula (I): 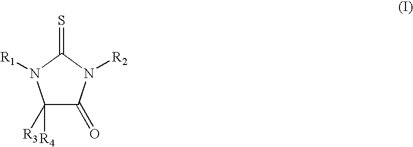
in which
R₁ and R₂ independently of one another are
a C₁-C₅ alkyl group,
a C₃-C₄ alkenyl group, or
a group —(CH₂)ₙ-Ar in which
n is 0 or 1, and
Ar is a phenyl ring that is unsubstituted or substituted by one or more atoms or groups of atoms selected from halogens, C₁-C₄ alkyl, nitro, C₁-C₃ alkoxy, methylenedioxy, carboxyl or alkoxy(C₁-C₄)carbonyl, and 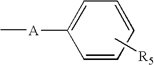
in which
A is CH₂O or OCH₂, and
R₅ is a hydrogen atom, a halogen atom, a C₁-C₄ alkyl group, a C₁-C₃ alkoxy group or a carboxyl or alkoxy(C₁-C₄)carbonyl group; and
R₃ and R₄ each independently are a hydrogen atom or a C₁-C₄ alkyl group,
with the proviso that at least one of the substituents R₁ and R₂ comprises 2 aromatic rings in its structure.

5. Compound according to claim 4, which is selected from the compounds of formula (I): 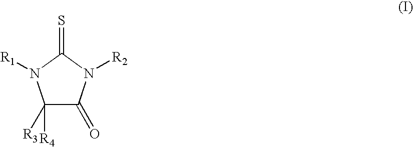
in which
R₁ is
a C₃-C₄ alkenyl group, or
a group —(CH₂)ₙ-Ar in which
n is 0 or 1, and
Ar is a phenyl ring that is unsubstituted or substituted by one or more atoms or groups of atoms selected from halogens, C₁-C₄ alkyl, nitro, C₁-C₃ alkoxy, carboxyl or alkoxy(C₁-C₄)carbonyl, and 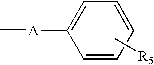
in which
A is CH₂O or OCH₂, and
R₅ is a hydrogen atom, a halogen atom, a C₁-C₄ alkyl group, a C₁-C₃ alkoxy group or a carboxyl or alkoxy(C₁-C₄)carbonyl group;
R₂ is
a C₁-C₅ alkyl group,
a C₃-C₄ alkenyl group, or
a group -Ar in which
Ar is a phenyl ring that is unsubstituted or substituted by one or more atoms or groups of atoms selected from halogens, C₁-C₄ alkyl, nitro, C₁-C₃ alkoxy, methylenedioxy, carboxyl or alkoxy(C₁-C₄)carbonyl, and 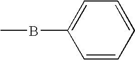
in which
B is CH₂O or OCH₂; and
R₃ and R₄ each independently are a hydrogen atom or a C₁-C₄ alkyl group,
with the proviso that at least one of the substituents R₁ and R₂ comprises 2 aromatic rings in its structure.

6. Compound derived from 2-thiohydantoin, characterized in that it which is selected from:
a) the compounds of formula (I): 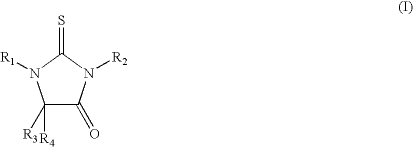
in which
R₁ and R₂ independently of one another are
a C₁-C₅ alkyl group,
a C₃-C₄ alkenyl group,
a C₂-C₃ hydroxyalkyl group or one of its precursors,
a C₃-C₅ alkoxyalkyl group, or
a group (CH₂)ₚ-Ar in which
p is 0 or 1, and
Ar is a phenyl or pyridinyl aromatic ring that is unsubstituted or substituted by one or more atoms or groups of atoms selected from halogens, hydroxyl, nitro, cyano, C₁-C₃ alkoxy, carboxyl, alkoxy(C₁-C₄)carbonyl, methylthio, methylenedioxy and 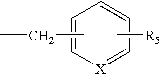
in which
X is CH or N, and
R₅ is a hydrogen atom, a halogen atom, a C₁-C₃ alkoxy group or a hydroxyl group; and
R₃ and R₄ each independently are a hydrogen atom or a C₁-C₄ alkyl group,
with the proviso that at least one of the substituents R₁ and R₂ comprises in its structure 2 aromatic rings selected from phenyl and pyridinyl groups; and
b) addition salts of the compounds of formula (I) with a non-toxic acid if said compounds of formula (I) comprise a salifiable basic group.

7. Compound according to claim 6, which is selected from:
a) the compounds of formula (I): 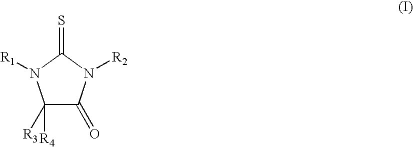
in which
R₁ is
a C₃-C₄ alkenyl group, or
a group (CH₂)ₙ-Ar in which
n is 0 or 1, and
Ar is a phenyl aromatic ring that is unsubstituted or substituted by one or more atoms or groups of atoms selected from halogens, C₁-C₃ alkoxy, nitro and the group 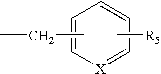
in which
X is CH or N, and
R₅ is a hydrogen atom, a halogen atom, a C₁-C₃ alkoxy group or a hydroxyl group;
R₂ is
a C₃-C₅ alkyl group,
a C₃-C₄ alkenyl group,
a C₂-C₃ hydroxyalkyl group or one of its precursors,
a C₃-C₅ alkoxyalkyl group, or
a group (CH₂)ₚ-Ar in which
p is 0 or 1, and
Ar is a phenyl or pyridinyl aromatic ring that is unsubstituted or substituted by one or more atoms or groups of atoms selected from halogens, hydroxyl, nitro, cyano, C₁-C₃ alkoxy, carboxyl, alkoxy(C₁-C₄)carbonyl, methylthio, methylenedioxy and 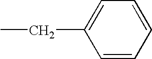
and
R₃ and R₄ each independently are a hydrogen atom or a C₁-C₄ alkyl group,
with the proviso that at least one of the substituents R₁ and R₂ comprises in its structure 2 aromatic rings selected from phenyl and pyridinyl groups; and
b) addition salts of the compounds of formula (I) with a non-toxic acid if said compounds of formula (I) comprise a salifiable basic group.

8. Compound according to claim 1, in which one of the radicals R₁ or R₂ is the phenoxyphenyl, phenylthiophenyl, (phenylmethoxy)phenyl or (phenylmethyl)phenyl group and the radicals R₃ and R₄ and the other radical R₁ or R₂ are as defined in claim 1.

9. Compound according to claim 2, in which one of the radicals R₁ or R₂ is the phenoxyphenyl or phenylthiophenyl group and the radicals R₃ and R₄ and the other radical R₁ or R₂ are as defined in claim 2.

10. Compound according to claim 4, in which one of the radicals R₁ or R₂ is the phenoxyphenyl, phenylthiophenyl, (phenylmethoxy)phenyl or (phenylmethyl)phenyl group and the radicals R₃ and R₄ and the other radical R₁ or R₂ are as defined in claim 4.

11. Compound according to claim 6, in which one of the radicals R₁ or R₂ is the phenoxyphenyl, phenylthiophenyl, (phenylmethoxy)phenyl or (phenylmethyl)phenyl group and the radicals R₃ and R₄ and the other radical R₁ or R₂ are as defined in claim 6.

12. Compound of formula (I) according to claim 1, in which R₃ is a methyl group and R₄ is a hydrogen atom or a methyl group.

13. Process for the preparation of a compound of formula (I) according to claim 1, wherein it comprises steps which consist in:
a) reacting an acid of the formula 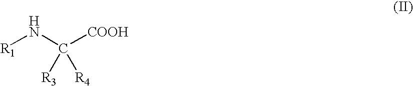
in which R₁ and R₄ are as defined above in claim 1 and R₃ is H, C₁-C₄ alkyl, phenyl or benzyl, with an isothiocyanate of formula (III):
R₂—N═C═S (III)
in which R₂ is a group as defined above in claim 1, in a solvent, at a temperature between 20° C. and the boiling point of the solvent, in the presence of a base, for 1 to 20 hours, to give the compound of formula (I): 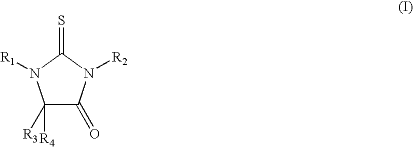
in which R₁, R₂, R₃ and R₄ are as defined for the starting materials; and
b) if necessary, if the compound of formula (I) obtained above contains a salifiable basic group such as an amine, reacting said compound with a mineral or organic acid, in an anhydrous solvent, to give the salt of the compound of formula (I).

14. Process for the preparation of a compound of formula (I) according to claim 1, wherein it consists in:
a) reacting an ester of formula (IV): 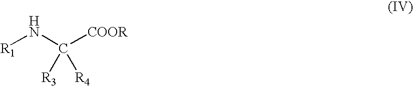
in which R₁ and R₄ are as defined in claim 1, R₃ is H, C₁-C₄ alkyl, phenyl or benzyl and R is a C₁-C₄ alkyl group, preferably a methyl, ethyl or isopropyl group, with an isothiocyanate of formula (III):
R₂—N═C═S (III)
the reaction being carried out in a solvent, in the presence of a weak acid, at a temperature between 80° C. and the boiling point of the solvent, for 0.5 to 5 hours, to give the compound of formula (I): 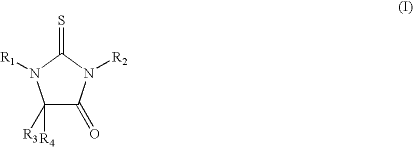
in which
R₁, R₂, R₃ and R₄ are as defined for the starting compounds; and
b) if necessary, in the case where the compound of formula (I) comprises a salifiable basic group, reacting said compound with an acid to give the corresponding salt.

15. Pharmaceutical composition, which it contains at least one compound of formula (I) according to claim 1 in association with at least one physiologically acceptable excipient.

16. Compound of formula (I) or one of its addition salts with a pharmaceutically acceptable acid, according to claim 1, as a pharmacologically active substance.

17. (canceled)

18. (canceled)

19. (canceled)

20. (canceled)

21. Method for the treatment of diabetes and diseases due to hyperglycemia, which consists in administering to a patient in need thereof an effective amount of a compound of formula (I) according to claim 1, or one of its addition salts with a pharmaceutically acceptable acid.

22. Method for the treatment of hypertriglyceridemia and dyslipidemia which consists in administering to a patient in need thereof an effective amount of a compound of formula (I) according to claim 1, or one of its addition salts with a pharmaceutically acceptable acid.

23. Method for the treatment of obesity which consists in administering to a patient in need thereof an effective amount of a compound of formula (I) according to claim 1, or one of its addition salts with a pharmaceutically acceptable acid.

24. Method for the treatment of cerebral vascular accidents which consists in administering to a patient in need thereof an effective amount of a compound of formula (I) according to claim 1, or one of its addition salts with a pharmaceutically acceptable acid.

25. Compound according to claim 3, in which one of the radicals R₁ or R₂ is the phenoxyphenyl or phenylthiophenyl group and the radicals R₃ and R₄ and the other radical R₁ or R₂ are as defined in claim 3.

26. Compound according to claim 5, in which one of the radicals R₁ or R₂ is the phenoxyphenyl, phenylthiophenyl, (phenylmethoxy)phenyl or (phenylmethyl)phenyl group and the radicals R₃ and R₄ and the other radical R₁ or R₂ are as defined in claim 5.

27. Compound according to claim 7, in which one of the radicals R₁ or R₂ is the phenoxyphenyl, phenylthiophenyl, (phenylmethoxy)phenyl or (phenylmethyl)phenyl group and the radicals R₃ and R₄ and the other radical R₁ or R₂ are as defined in claim 7.

28. Compound of formula (I) according to claim 2 in which R₃ is a methyl group and R₄ is a hydrogen atom or a methyl group.

29. Compound of formula (I) according to claim 3 in which R₃ is a methyl group and R₄ is a hydrogen atom or a methyl group.

30. Compound of formula (I) according to claim 4 in which R₃ is a methyl group and R₄ is a hydrogen atom or a methyl group.
